(19) United States
(12) Patent Application Publication
(10) Pub. No.: US 2017/0305861 A1
(43) Pub. Date: Oct. 26, 2017

(54) HETEROARYL COMPOUNDS COMPRISING NITROGEN AND USE THEREOF

(71) Applicant: ImmunoMet Therapeutics, Inc, Houston, TX (US)

(72) Inventors: Sung Wuk Kim, Seongnam-si (KR); Hong Woo Kim, Irvine, CA (US); SangHee Yoo, Houston, TX (US); Hyunwook Kim, Seoul (KR); Hye Jin Heo, Daejeon (KR); Hong Bum Lee, Daejeon (KR); Jiae Kook, Daejeon (KR); Young Woo Lee, Yangyang-gun (KR)

(73) Assignee: ImmunoMet Therapeutics, Inc, Houston, TX (US)

(21) Appl. No.: 15/496,631

(22) Filed: Apr. 25, 2017

Related U.S. Application Data
(60) Provisional application No. 62/327,249, filed on Apr. 25, 2016.

(51) Int. Cl. C07D 227/02; A61K 45/06; C07C 11/02; C07D 283/00

(52) U.S. Cl. CPC C07D 227/02; C07D 283/00; A61K 45/06; C07C 11/02

(57) ABSTRACT

The present invention relates to heteroaryl compounds comprising nitrogen and use thereof, and more specifically to compounds which exhibit a remarkable effect on inhibiting proliferation of cancer cells and metastasis and recurrence of cancer, a preparation method of the same, and a pharmaceutical composition comprising the same as an active ingredient.

The compounds according to the present invention exhibit a remarkable effect on inhibiting proliferation of cancer cells and metastasis and recurrence of cancer with a reduced dose compared to that of existing drugs. Accordingly, the compounds can be effectively used for treating various types of cancer, such as uterine cancer, breast cancer, gastric cancer, brain cancer, rectal cancer, colorectal cancer, lung cancer, skin cancer, blood cancer, pancreatic cancer, renal cancer, prostate cancer, bladder cancer, and liver cancer, and for inhibiting proliferation of cancer cells and metastasis of cancer.

### TECHNICAL FIELD

The present invention relates to heteroaryl compounds comprising nitrogen and use thereof, and more specifically to heteroaryl compounds comprising nitrogen which exhibit a remarkable effect on inhibiting proliferation of cancer cells and delaying and inhibiting metastasis of cancer, a preparation method thereof, and a pharmaceutical composition comprising the same as an active ingredient.

### BACKGROUND ART

Normal cells with sufficient oxygen produce adenosine triphosphate (ATP) through oxidative phosphorylation while rarely producing lactate, whereas cancer cells produce ATP through glycolysis and fermentation of lactic acid. Accordingly, cancer cells require more glucose compared to normal cells. Further, even in an aerobic environment, cancer cells cause oncogenic metabolism where glucose prefers glycolysis. In this case, there is reportedly a marked increase in mitochondrial membrane potential. Cancer cells use such metabolic pathway as a main energy supply source to generate energy, and construct an environment which activates survival, proliferation, angiogenesis, and metastasis of cancer cells, thereby resulting in the progression of a malignant tumor. Therefore, inhibiting such mitochondrial function and energy metabolism of cancer cells is highly likely to solve the problem in which existing targeting anti-cancer agents have narrow therapeutic regions and resistance issues, and there is currently considerable interest in developing anti-cancer agents targeting such metabolic characteristics of cancer cells (Nat Rev Cancer. 2011; 11: 85-95).

Berberine is a type of alkaloid with 4 substituents on a positively charged ammonium ion and an alkyl or aryl group on the R group. Berberine reportedly blocks growth pathways of cancer cells (Carcinogenesis. 2011; 86-92, Anticancer Res. 2009; 4063-4070), or regulates intracellular energy metabolism by inhibiting complex 1 in mitochondria and oxidative phosphorylation. Accordingly, berberine is known as exhibiting an anti-cancer effect by inhibiting differentiation and survival of cancer cells, and killing cancer stem cells (Diabetes. 2008; 1414-1418, J. Pharmacol. Exp. Ther. 2007; 636-649). Further, research results indicating that berberine inhibits growth of lung cancer cell lines and epithelial-to-mesenchymal transition (EMT) of cancer cells (J Transl Med. 2014; 12: 22) suggest that berberine has potential as a metastasis inhibitor. Additionally, research on therapies by combined use of berberine with other compounds has been actively conducted, which suggests that berberine has potential as a chemotherapeutic agent. However, low concentration of berberine in the blood implies the possibility of problematic overdose thereof (Metabolism. 2010; 285-292). Therefore, novel drugs through synthesis of heteroaryl compounds comprising nitrogen are being developed so as to maintain pharmaceutical significance of a berberine compound, to enhance in vivo absorbability of the same by complementing the defect of the low concentration in the blood, and to induce the effect of combined use with existing anti-cancer agents.

### DISCLOSURE

### Technical Problem

The present invention provides heteroaryl compounds comprising nitrogen which exhibit a remarkable effect on inhibiting proliferation of cancer cells and metastasis and recurrence of cancer with a smaller dose than that of existing drugs, a pharmaceutically acceptable salt thereof, and a preparation method of the same.

Additionally, the present invention provides a pharmaceutical composition for treating cancer comprising the compound or a pharmaceutically acceptable salt thereof. Specifically, the cancer may be a disease selected from the group consisting of prostate cancer, uterine cancer, breast cancer, gastric cancer, brain cancer, rectal cancer, colorectal cancer, lung cancer, skin cancer, blood cancer, pancreatic cancer, renal cancer, bladder cancer, prostate cancer, and liver cancer.

### Technical Solution

In order to solve the aforementioned technical problems, an embodiment of the present invention provides a compound represented by Formula 1 below which exhibits a remarkable effect on inhibiting proliferation of cancer cells and metastasis and recurrence of cancer with a reduced dose compared to that of existing drugs, a pharmaceutically acceptable salt thereof, and a preparation method of the same.

In addition, it provides a pharmaceutical composition comprising the compound represented by Formula 1 and a pharmaceutically acceptable salt thereof and also provides a method for treating or preventing cancer, comprising administering a therapeutically effective amount of the same to a subject in need thereof.

The present invention provides a compound represented by Formula 1 below and a pharmaceutically acceptable salt thereof.

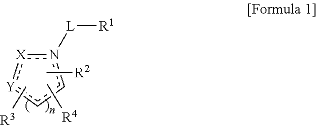

In Formula 1,

[symbol] refers to a single bond or double bond, and a ring of Formula 1 comprises two to three double bonds, wherein the double bonds are not adjacent to each other,

X is CH, CNH₂, or N,

Y is CH, N, or S,

n is 1 or 2,

L is C₁₋₆ alkylene or C₁₋₆ alkenylene,

R¹ is C₆₋₁₄ aryl, C₅₋₂₀ heteroaryl, C₃₋₈ cycloalkyl, or C₃₋₈ heterocycloalkyl, and

R² to R⁴ are each independently hydrogen, amino (—NH₂), substituted amino (—NHR′ or —NR′R″), nitro, halogen, cyano, oxo, hydroxy, C₁₋₆ alkyl, C₃₋₈ cycloalkyl, C₃₋₈ heterocycloalkyl, C₁₋₆ alkoxy, C₁₋₆ haloalkyl, or C₁₋₆ haloalkoxy; or R² and R³ are positioned on adjacent carbon atoms and connected to each other to form a ring,

wherein R′ and R″ are each independently C₁₋₆ alkyl; or R′ and R″ are connected to each other to form a ring comprising a nitrogen atom to which R′ and R″ are bonded.

As used herein, the term “alkylene” refers to a bivalent functional group derived from alkane, and “alkenylene” refers to a bivalent functional group derived from alkene.

As used herein, the term “aryl” refers to a fused or unfused mono- or poly-cyclic carbocyclic ring system having at least one aromatic ring, but is not limited to, including phenyl, naphthyl, tetrahydronaphthyl, indanyl, indenyl, etc.

As used herein, the term “heteroaryl” refers to a mono- or poly-cyclic (e.g., bi-, tri-cyclic, or higher) fused or unfused part or ring system, having at least one aromatic ring, and having 5 to 20 ring atoms wherein one of the ring atoms is selected from S, O, Se, and N; 0, 1, or 2 ring atoms are additional heteroatoms independently selected from S, O, Se, and N; and further, the rest of the ring atoms are carbon. Heteroaryl includes, but is not limited to, pyridinyl, pyrazinyl, pyrimidinyl, pyrrolyl, pyrazolyl, imidazolyl, thiazolyl, oxazolyl, isoxazolyl, thiadiazolyl, oxadiazolyl, thiophenyl, furanyl, quinolinyl, isoquinolinyl, benzimidazolyl, benzoxazolyl, quinoxalinyl, etc.

As used herein, the term “cycloalkyl” refers to a monovalent group derived from a monocyclic or polycyclic saturated or partially unsaturated carbocyclic ring compound.

Examples of C₃-C₁₀-cycloalkyl include, but are not limited to, cyclopropyl, cyclobutyl, cyclopentyl, cyclohexyl, cyclopentyl, and cyclooctyl, and further, examples of C₃-C₁₂-cycloalkyl include, but are not limited to, cyclopropyl, cyclobutyl, cyclopentyl, cyclohexyl, bicyclo[2.2.1]hexyl, and bicyclo[2.2.2.]octyl. Further, a monovalent group, derived from a monocyclic or polycyclic carbocyclic ring having at least one carbon-carbon double bond by the removal of a single hydrogen atom, is considered.

As used herein, the term “heterocycloalkyl” refers to a non-aromatic 3-, 4-, 5-, 6-, or 7-membered ring or bi- or tri-cyclic group fused or unfused system, and in particular, (i) each ring contains 1 to 3 heteroatoms independently selected from oxygen, sulfur, and nitrogen, (ii) each 5-membered ring has 0 to 1 double bonds, and each 6-membered ring has 0 to 2 double bonds, iii) nitrogen and sulfur heteroatoms may optionally be oxidized, (iv) nitrogen heteroatom may optionally be quaternized, and (iv) any of the rings may be fused to a benzene ring. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thaizolidinyl, isothiazolidinyl, and tetrahydrofuryl.

As used herein, the term “oxo” preferably refers to oxygen attached to carbon by a double bond (e.g., carbonyl).

As used herein, the term “alkyl” refers to saturated, straight, or branched hydrocarbon moieties each containing 1 to 6 or 1 to 8 hydrocarbons in certain embodiments. Examples of C₁ to C₆ moieties include, but are not limited to, methyl, ethyl, propyl, isopropyl, n-butyl, tert-butyl, neopentyl, n-hexyl moieties; and further, examples of C₁ to C₈ moieties include, but are not limited to, methyl, ethyl, propyl, isopropyl, n-butyl, tert-butyl, neopentyl, n-hexyl, hexyl, and octyl moieties.

As used herein, the term “alkoxy” refers to —O-alkyl moieties.

As used herein, the terms “halo” and “halogen” refer to an atom selected from fluoro, chloro, bromo, and iodo.

Specifically, the compound represented by Formula 1 above may be a compound in which heteroaryl comprising nitrogen is linked with a cyclic compound by a linker (L).

A linker (L) may be C₁₋₆ alkylene or C₁₋₆ alkenylene which is unsubstituted or substituted with oxo, and specifically may be C₁₋₆ alkylene which is unsubstituted or substituted with oxo, and more specifically may be methylene, ethylene, propylene, or —CH₂—C(O)—.

R¹ may be C₆₋₁₄ aryl, C₅₋₂₀ heteroaryl, C₃₋₈ cycloalkyl, or C₃₋₈ heterocycloalkyl which is unsubstituted or substituted with one or more substituents selected from the group consisting of hydroxy, halogen, amino, cyano, nitro, C₁₋₆ alkyl, C₁₋₆ alkoxy, C₁₋₉₆ haloalkyl, and C₁₋₆ haloalkoxy. Specifically, R¹ may be C₆₋₈ aryl, C₃₋₈ cycloalkyl, or C₅₋₈ heteroaryl which is unsubstituted or substituted with halogen, C₁₋₆ haloalkoxy, or C₁₋₆ alkyl, and more specifically, may be C₆₋₈ aryl, C₃₋₈ cycloalkyl, or C₅₋₈ heteroaryl which is unsubstituted or substituted with chlorine, fluorine, trifluoromethoxy, or methyl. More specifically, R¹ may be phenyl, cyclopropyl, cyclobutyl, cyclopentyl, cyclohexyl, thiophene, furan, or selenophene which is unsubstituted or substituted with chlorine, fluorine, trifluoromethoxy, or methyl.

R² to R⁴ may be C₁₋₆ alkyl, C₃₋₈ cycloalkyl, C₃₋₈ heterocycloalkyl, C₁₋₆ alkoxy, C₁₋₆ haloalkyl, or C₁₋₆ haloalkoxy which is each independently unsubstituted or substituted with one or more substituents selected from the group consisting of halogen, hydroxy, cyano, nitro, C₁₋₆ alkyl, C₁₋₆ alkoxy, C₁₋₆ haloalkyl, and C₁₋₆ haloalkoxy, and specifically, may be hydrogen, amino (—NH₂), substituted amino (—NHR′ or —NR′R″), oxo, nitro, halogen, cyclopropyl, methyl, methoxy, ethoxy, or isopropoxy which is unsubstituted or substituted with one or more substituents selected from the group consisting of halogen, hydroxy, cyano, nitro, C₁₋₆ alkyl, C₁₋₆ alkoxy, C₁₋₆ haloalkyl, and C₁₋₆ haloalkoxy.

In addition, the ring formed by R² and R³ being positioned on adjacent carbon atoms and connected to each other is substituted with one or more substituents selected from the group consisting of halogen, hydroxy, cyano, nitro, C₁₋₆ alkyl, C₁₋₆ alkoxy, C₁₋₆ haloalkyl, and C₁₋₆ haloalkoxy.

The ring formed by R² and R³ being positioned on adjacent carbon atoms and connected to each other may be C₆ to C₁₄ aryl, C₅ to C₂₀ heteroaryl, C₃ to C₁₀ cycloalkyl, or C₃ to C₁₀ heterocycloalkyl, and specifically, C₃ to C₁₀ cycloalkyl formed by R² and R³ being positioned on adjacent carbon atoms and connected to each other may be cyclohexyl, C₃ to C₁₀ heterocycloalkyl formed by R² and R³ being positioned on adjacent carbon atoms and connected to each other may be piperidinyl or morpholinyl, and C₆ to C₈ aryl formed by R² and R³ being positioned on adjacent carbon atoms and connected to each other may be benzo.

R′, R″, or, when R′ and R″ are connected to each other to form a ring comprising a nitrogen atom to which R′ and R″ are bonded, the ring may each be independently substituted with one or more substituents selected from the group consisting of hydroxy, cyano, nitro, C₁₋₆ alkyl, C₁₋₆ alkoxy, C₁₋₆ haloalkyl, and C₁₋₆ haloalkoxy.

R′ and R″ may each independently be C₁₋₆ alkyl, and specifically, R′ and R″ may each independently be methyl, tertiary butyl,

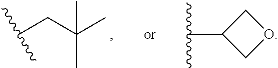

The ring formed by R′ and R″ being connected to each other comprising a nitrogen atom to which R′ and R″ are bonded may be C₆ to C₁₄ aryl, C₅ to C₂₀ heteroaryl, C₃ to C₁₀ cycloalkyl, or C₃ to C₁₀ heterocycloalkyl, and specifically, may be C₃ to C₁₀ heterocycloalkyl, and more specifically, may be morpholinyl, azetidinyl, pyrrolidinyl, piperidinyl, or azepanyl which is unsubstituted or substituted with one or more halogens.

In the present invention, the compound represented by Formula 1 above or a pharmaceutically acceptable salt thereof may specifically be the following compound.

| | |
|---|---|
| Example 1 | 4-amino-1-phenethylpyridinium chloride |
| Example 2 | 4-nitro-1-phenethyl-1H-imidazole |
| Example 3 | 4-nitro-1-phenethyl-1H-imidazole hydrochloride |
| Example 4 | 3-nitro-1-phenethyl-1H-pyrazole |
| Example 5 | 1-phenethyl-1H-pyrazol-3-amine |
| Example 6 | 6-amino-3-phenethylpyrimidin-4(3H)-one |
| Example 7 | 4-amino-2-bromo-1-phenethylpyridinium chloride |
| Example 8 | 2,4-diamino-1-phenethylpyridinium bromide |
| Example 9 | 1-phenethyl-1H-imidazole |
| Example 10 | 2,6-diamino-3-phenethylpyrimidin-4(3H)-one |
| Example 11 | 4-amino-1-(2-chlorophenethyl)pyridinium chloride |
| Example 12 | 2,4-diamino-1-(2-chlorophenethyl)pyridinium bromide |
| Example 13 | 3-phenethylthiazol-3-ium iodide |
| Example 14 | 2-amino-3-phenethylthiazol-3-ium iodide |
| Example 15 | 4-amino-2-cyclopropyl-1-phenethylpyridinium iodide |
| Example 16 | 4-amino-1-phenethylquinolinium iodide |
| Example 17 | 4-(dimethylamino)-1-phenethylpyridinium chloride |
| Example 18 | 4-amino-2-fluoro-1-phenethylpyridinium chloride |
| Example 19 | 4-amino-1-(3,4-dichlorophenethyl)pyridinium chloride |
| Example 20 | 4-amino-1-benzylpyridinium chloride |
| Example 21 | 4-amino-1-benzyl-2-fluoropyridinium chloride |
| Example 22 | 1-phenethyl-5,6,7,8-tetrahydroquinolinium chloride |
| Example 23 | 4-amino-1-(3-phenylpropyl)pyridinium chloride |
| Example 24 | 4-amino-2-fluoro-1-(3-phenylpropyl)pyridinium chloride |
| Example 25 | 4-amino-1-(2-oxo-2-(4-(trifluoromethoxy)phenyl)ethyl)pyridinium bromide |
| Example 26 | 4-amino-1-(2-oxo-2-phenylethyl)pyridinium bromide |
| Example 27 | 4-amino-1-(2-cyclohexylethyl)pyridinium bromide |
| Example 28 | 4-amino-1-(2-cyclohexylethyl)-2-fluoropyridinium bromide |
| Example 29 | 2,4-diamino-1-benzylpyridinium chloride |
| Example 30 | 4-amino-1-benzyl-2-chloropyridinium chloride |
| Example 31 | 4-amino-1-(cyclopropylmethyl)pyridinium chloride |
| Example 32 | 4-amino-2-chloro-1-phenethylpyridinium chloride |
| Example 33 | 4-(methylamino)-1-phenethylpyridinium chloride |
| Example 34 | 1-benzyl-4-(methylamino)pyridinium chloride |
| Example 35 | 4-amino-1-(3,4-dichlorobenzyl)-2-fluoropyridinium chloride |
| Example 36 | 4-amino-1-(3,4-dichlorobenzyl)pyridinium chloride |
| Example 37 | 1-(3,4-dichlorobenzy])-4-(methylamino)pyridinium chloride |
| Example 38 | 1-(3,4-dichlorobenzy])-4-(dimethylamino)pyridinium chloride |
| Example 39 | 4-amino-1-(cyclopropylmethyl)-2-fluoropyridinium chloride |
| Example 40 | 2,4-diamino-1-(2-cyclohexylethyl)pyridinium bromide |
| Example 41 | 1-(cyclopropylmethyl)-4-(methylamino)pyridinium chloride |
| Example 42 | 1-(cyclopropylmethyl)-4-(dimethylamino)pyridinium chloride |
| Example 43 | 4-amino-3-methyl-1-benzylpyridinium chloride |
| Example 44 | 4-amino-3-methyl-1-phenethylpyridinium chloride |
| Example 45 | 4-amino-1-benzyl-2-methoxypyridinium chloride |
| Example 46 | 4-amino-1-(cyclohexylmethyl)pyridinium bromide |
| Example 47 | 4-amino-1-(cyclobutylmethyl)pyridinium bromide |
| Example 48 | 4-amino-1-(cyclobutylmethyl)-2-fluoropyridinium bromide |
| Example 49 | 4-amino-1-(4-fluorobenzyl)pyridinium bromide |
| Example 50 | 1-benzyl-4-morpholinopyridinium chloride |
| Example 51 | 4-morpholino-1-phenethylpyridinium chloride |
| Example 52 | 4-morpholino-1-(cyclopropylmethyl)pyridinium chloride |
| Example 53 | 1-(2-cyclohexylethyl)-4-morpholinopyridinium bromide |
| Example 54 | 1-benzyl-4-(pyrrolidin-1-yl)pyridinium chloride |
| Example 55 | 1-phenethyl-4-(pyrrolidin-1-yl)pyridinium chloride |
| Example 56 | 1-(cyclopropylmethyl)-4-(pyrrolidin-1-yl)pyridinium chloride |
| Example 57 | 1-(cyclohexylmethyl)-4-(pyrrolidin-1-yl)pyridinium bromide |
| Example 58 | 1-(cyclobutylmethyl)-4-(pyrrolidin-1-yl)pyridinium chloride |
| Example 59 | 1-benzyl-4-(piperidin-1-yl)pyridimum chloride |
| Example 60 | 4-(azepan-1-yl)-1-benzylpyridinium chloride |
| Example 61 | 1-benzyl-4-(neopentylamino)pyridinium chloride |
| Example 62 | 4-(pyrrolidin-1-yl)-1-(thiophen-3-ylmethyl)pyridinium bromide |
| Example 63 | 6-(cyclopropylmethyl)-1,2,3,4-tetrahydro-1,6-naphthyridin-6-ium chloride |
| Example 64 | 6-(cyclopropylmethyl)-2,3-dihydro-1H-pyrido[3,4-b][1,4]oxazin-6-ium chloride |
| Example 65 | 1-benzyl-4-(4,4-difluoropiperidin-1-yl)pyridinium chloride |
| Example 66 | 4-(azetidin-1-yl)-1-benzylpyridinium chloride |
| Example 67 | 1-benzyl-4-(oxetan-3-ylamino)pyridinium chloride |
| Example 68 | 4-(pyrrolidin-1-yl)-1-(thiophen-2-ylmethyl)pyridinium chloride |
| Example 69 | 1-benzyl-4-(tert-butylamino)pyridinium chloride |
| Example 70 | 4-(azetidin-1-yl)-1-(cyclopropylmethyl)pyridinium chloride |
| Example 71 | 4-(azetidin-1-yl)-1-(thiophen-3-ylmethyl)pyridinium bromide |
| Example 72 | 4-(pyrrolidin-1-yl)-1-(selenophen-2-ylmethyl)pyridinium chloride |
| Example 73 | 4-amino-1-(cyclopropylmethyl)pyrimidin-1-ium chloride |
| Example 74 | 4-amino-1-(selenophen-2-ylmethyl)pyrimidin-1-ium chloride |
| Example 75 | 4-amino-1-(selenophen-2-ylmethyl)pyridazin-1-ium chloride |
| Example 76 | 4-amino-1-(selenophen-2-ylmethyl)pyridinium chloride |
| Example 77 | 4-amino-1-(thiophen-2-ylmethyl)pyridinium chloride |
| Example 78 | 1-(furan-2-ylmethyl)-4-(pyrrolidin-1-yl)pyridinium chloride |
| Example 79 | 1-((5-methylthiophen-2-yl)methyl)-4-(pyrrolidin-1-yl)pyridinium chloride |
| Example 80 | 4-(azetidin-1-yl)-1-(selenophen-3-ylmethyl)pyridinium chloride |
| Example 81 | 2-amino-4-(azetidin-1-yl)-1-(cyclopropylmethyl)pyridinium chloride |
| Example 82 | 2-amino-4-(azetidin-1-yl)-1-(cyclopropylmethyl)pyridinium chloride |
| Example 83 | 2,4-diamino-1-(cyclopropylmethyl)pyridinium chloride |
| Example 84 | 2,4-diamino-1-(4-chlorobenzyl)pyridinium chloride |
| Example 85 | 2-amino-4-(azetidin-1-yl)-1-((5-methylthiophen-2-yl)methyl)pyridinium chloride |
| Example 86 | 2-amino-4-(azetidin-1-yl)-1-(selenophen-2-ylmethyl)pyridinium chloride |
| Example 87 | 2-amino-4-(azetidin-1-yl)-1-benzylpyridinium chloride |
| Example 88 | 2-amino-1-benzyl-4-(pyrrolidin-1-yl)pyridinium chloride |
| Example 89 | 2-amino-1-(cyclopropylmethyl)-4-(pyrrolidin-1-yl)pyridinium chloride |
| Example 90 | 2-amino-1-((5-methylthiophen-2-yl)methyl)-4-(pyrrolidin-1-yl)pyridinium chloride |
| Example 91 | 2-amino-4-(pyrrolidin-1-yl)-1-(selenophen-2-ylmethyl)pyridinium chloride |
| Example 92 | 2-amino-1-(4-chlorobenzyl)-4-(pyrrolidin-1-yl)pyridinium chloride |
| Example 93 | 4-amino-1-benzyl-2-ethoxypyridinium chloride |
| Example 94 | 4-amino-1-benzyl-2-isopropoxypyridinium chloride |
| Example 95 | 4-amino-1-benzyl-2-cyclopropylpyridinium chloride |
| Example 96 | 4-(azetidin-1-yl)-1-benzyl-2-ethoxypyridinium chloride |
| Example 97 | 4-(azetidin-1-yl)-1-benzyl-2-isopropoxypyridinium chloride |
| Example 98 | 4-(azetidin-1-yl)-1-benzyl-2-cyclopropylpyridinium chloride |
| Example 99 | 1-benzyl-2-ethoxy-4-(pyrrolidin-1-yl)pyridinium chloride |
| Example 100 | 1-benzyl-2-isopropoxy-4-(pyrrolidin-1-yl)pyridinium chloride |
| Example 101 | 1-benzy1-2-cyclopropyl-4-(pyrrolidin-1-yl)pyridinium chloride |

The present invention provides a method for preparing the compound of Formula 1 or a pharmaceutically acceptable salt thereof according to the present invention, comprising reacting a compound represented by Formula 2 below and a compound represented by Formula 3 below.

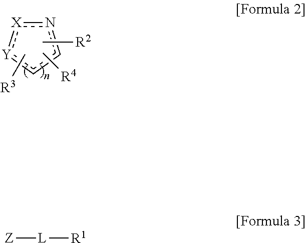

In Formulas 2 and 3,

Z is halogen, and [symbol] , X, Y, n, L, R¹, R², R³, and R⁴ are the same as defined above.

Z may specifically be chlorine.

A step of reacting the compound represented by Formula 2 and the compound represented by Formula 3 in the preparation method may be performed in an organic solvent, and the organic solvent may be dimethylformamide (DMF). The step may be performed at a temperature of 70° C. to 120° C., and may be performed for 3 hours to 12 hours.

The preparation method may further include a step of cooling a reaction solution to room temperature, a step of solidifying the product by adding an antisolvent to the solution, and a step of filtering the solidified product. The preparation method may further include a purification step, and specifically, may include a step of adding alcohol and drying the product under reduced pressure. The antisolvent may be diethyl ether, and the alcohol may be methanol.

The preparation method may further include a step of modifying the substituent of R² to R⁴, and in one exemplary embodiment, 4-amino-2-bromo-1-phenethylpyridinium chloride can be reacted with NH₄OH to produce 2,4-diamino-1-phenethylpyridinium bromide. In another exemplary embodiment, a hydrogen gas can be added to 4-nitro-1-phenethyl-1H-pyrazole hydrochloride under Pd/C to produce 1-phenethyl-1H-pyrazol-3-amine.

One exemplary embodiment of the preparation method is as follows.

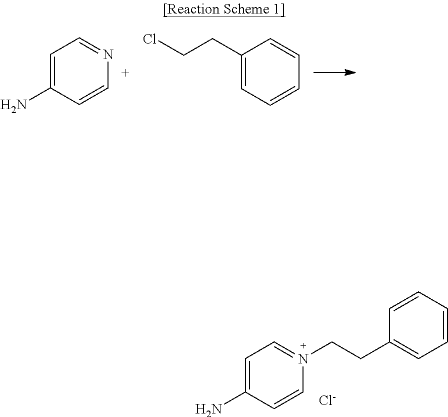

4-Aminopyridine and 2-chloroethylbenzene are added, and the mixture is stirred at 90° C. for 5 hours. After the reaction is completed, the solid resultant is filtered to obtain the compound.

Meanwhile, the pharmaceutically acceptable salt of the above compound of the present invention may be an acid addition salt formed using organic or inorganic acid. Examples of the organic acid include formic acid, acetic acid, propionic acid, lactic acid, butyric acid, isobutyric acid, trifluoroacetic acid, malic acid, maleic acid, malonic acid, fumaric acid, succinic acid, succinic acid monoamide, glutamic acid, tartaric acid, oxalic acid, citric acid, glycolic acid, glucuronic acid, ascorbic acid, benzoic acid, phthalic acid, salicylic acid, anthranilic acid, dichloroacetic acid, aminooxyacetic acid, benzene sulfonic acid, 4-toluene sulfonic acid, methanesulfonic acid, and salts thereof, and examples of the inorganic acid include hydrochloric acid, hydrobromic acid, sulfuric acid, phosphoric acid, nitric acid, carbonic acid, boric acid, and salts thereof. The above-mentioned acid addition salts may be prepared by a general method of preparing a salt, including a) directly mixing the compound of Formula a and an acid, b) dissolving one of the compound and an acid in a solvent or a hydrated solvent and mixing the resulting solution, or c) dissolving the compound of Formula 1 and an acid in a solvent or a hydrated solvent, and mixing them.

In one specific embodiment, the pharmaceutically acceptable salt of the compound may be a salt with an acid selected from the group consisting of formic acid, acetic acid, propionic acid, lactic acid, butyric acid, isobutyric acid, trifluoroacetic acid, malic acid, maleic acid, malonic acid, fumaric acid, succinic acid, succinic acid monoamide, glutamic acid, tartaric acid, oxalic acid, citric acid, glycolic acid, glucuronic acid, ascorbic acid, benzoic acid, phthalic acid, salicylic acid, anthranilic acid, benzenesulfonic acid, p-toluenesulfonic acid, methanesulfonic acid, dichloroacetic acid, aminooxyacetic acid, hydrochloric acid, hydrobromic acid, sulfuric acid, phosphoric acid, nitric acid, carbonic acid, and boric acid.

Therefore, an additional embodiment of the present invention provides a pharmaceutical composition comprising the compound of Formula 1 of the present invention or a pharmaceutically acceptable salt thereof as an active ingredient. The pharmaceutical composition of the present invention has a remarkable effect on proliferation of cancer cells, and may be used as an anti-cancer agent for various cancers, which specifically include uterine cancer, breast cancer, gastric cancer, brain cancer, rectal cancer, colorectal cancer, lung cancer, skin cancer, blood cancer, pancreatic cancer, renal cancer, bladder cancer, prostate cancer, liver cancer, etc., but are not limited thereto.

The pharmaceutical composition of the present invention may include at least one type of pharmaceutically acceptable carrier in addition to an active ingredient. As used herein, the term “pharmaceutically acceptable carrier” in the present invention refers to a disclosed pharmaceutical excipient which is useful upon formulation of a pharmaceutically active compound for administration, and which is substantially nontoxic and non-sensitive under conditions in use. An exact ratio of such excipient is determined by pharmaceutical standard practices as well as solubility, chemical properties, and selected administration routes of an active compound.

The pharmaceutical composition of the present invention may be formulated in a form which is suitable for a desired administration method by using an adjuvant, such as an excipient, disintegrating agent, sweetening agent, bonding agent, coating agent, inflating agent, lubricant, glydent, and flavoring agent.

The pharmaceutical composition may be formulated in a form of a tablet, capsule, pill, granule, powder, injection, or liquid, but is not limited thereto.

A formulation of the pharmaceutical composition and a pharmaceutically acceptable carrier may be appropriately selected by techniques disclosed in the art.

The pharmaceutical composition of the present invention may further comprise an anti-cancer agent, and specifically, may further comprise berberine.

Meanwhile, as used herein, the term “subject” refers to a warm-blooded animal such as a mammal with a specific disease, disorder, or condition. Examples thereof include humans, orangutans, chimpanzees, mice, rats, dogs, cows, chickens, pigs, goats, sheep, etc., but are not limited thereto.

In addition, the term “prevention” means all actions that inhibit disease or delay its progress.

As used herein, the term “treatment” includes amelioration of a symptom, temporary or perpetual removal of a symptomatic source, and prevention or slowdown of presence of a symptom and progress of the above-mentioned disease, disorder, or condition, but is not limited thereto.

A therapeutically effective amount of an active ingredient of the pharmaceutical composition in the present invention refers to an amount which is required for treatment of a condition. In this regard, the amount may be adjusted by various factors, such as condition types, severity of conditions, types and contents of effective and other ingredients contained in the composition, formulation types, patients' age, weight, general health condition, sex, and diet, administration time and route, release rate of the composition, treatment period, and concurrently used drugs. For example, in the case of adults, the compound of Formula 1 may be administered at a dose of 50 mg/kg to 3,000 mg/kg in total through one to multiple administrations per day.

### Advantageous Effects

The compounds of the present invention exhibit a remarkable effect on inhibiting proliferation of cancer cells and metastasis and recurrence of cancer with a smaller dose than that of existing drugs. Accordingly, the compounds can be effectively used for treating various cancer types, such as uterine cancer, breast cancer, gastric cancer, brain cancer, rectal cancer, colorectal cancer, lung cancer, skin cancer, blood cancer, pancreatic cancer, renal cancer, bladder cancer, prostate cancer, and liver cancer, and for inhibiting proliferation of cancer cells and metastasis of cancer.

### BRIEF DESCRIPTION OF THE DRAWINGS

FIG. 1 is the result of observing the volume of a tumor according to Test Example 3.

### DETAILED DESCRIPTION OF THE INVENTION

Hereinafter, the present invention will be described through Examples and Comparative Examples in more detail. However, the Examples disclosed herein are only for illustrative purposes, and should not be construed as limiting the scope of the present invention.

### Example 1: 4-Amino-1-phenethylpyridinium chloride

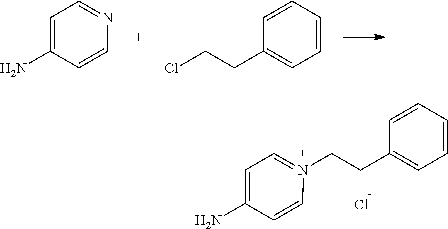

4-Aminopyridine (0.2 g, 2.12 mmol) was dissolved in DMF (5 mL) at room temperature. 2-Chloroethylbenzene (1.392 mL, 10.6 mmol) was added thereto, and the mixture was stirred for 5 hours at 90° C. After the reaction was completed, the mixture was cooled to room temperature, diethyl ether was added, and the mixture was stirred at room temperature for 30 minutes. The solid resultant was filtered. The obtained solid was dissolved in a small amount of methanol, ethyl acetate was added, and the mixture was stirred at room temperature for 1 hour. The formed solid was filtered and dried under reduced pressure to obtain a desired compound (86 mg, 17.2%).

¹H NMR (400 MHz, DMSO-D6) δ 8.29 (s, 2H), 8.10 (d. J=7.6 Hz, 2H), 7.31 (t, J=8.4 Hz, 2H), 7.249 (d, J=7.2 Hz, 1H), 7.20 (d, J=7.2 Hz, 2H), 6.81 (d, J=7.2 Hz, 2H), 4.37 (t J=6.8 Hz, 2H), 3.09 (t, J=6.8 Hz, 2H).

LCMS: 199.1 [M].

### Example 2: 4-Nitro-1-phenethyl-H-imidazole

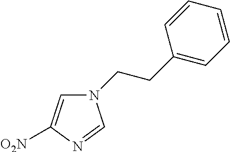 [SMILES: O=[N+]([O-])c1cn(CCc2ccccc2)cn1]

In the same manner as in Example 1, except that 4-nitro-1H-imidazole was used instead of 4-aminopyridine, 50 mg (11.1%) of a desired compound, which is a white solid, was obtained.

¹H NMR (400 MHz. DMSO-D6) δ 8.39 (d, J=1.6 Hz, 1H), 7.74 (d, J=1.2 Hz, 1H) 7.23 (m, 5H), 4.33 (t, J=7.2 Hz, 2H), 3.11 (t, J=7.6 Hz; 2H).

LCMS: 218.0 [M+H]⁺.

### Example 3: 4-Nitro-1-phenethyl-1H-imidazole hydrochloride

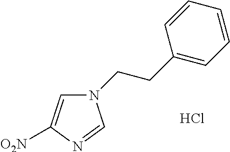 [SMILES: O=[N+]([O-])c1cn(CCc2ccccc2)cn1 ; Cl]

After dissolving the compound of Example 2 in methanol, 1 equivalent amount of 4 M HCl was added, and the mixture was stirred at room temperature for 1 hour. After concentration under reduced pressure, 50 mg of a desired compound, which is a white solid, was obtained.

¹H NMR (400 MHz, DMSO-D6) δ 8.39 (d, J=1.6 Hz, 1H), 7.74 (d, J=1.2 Hz, 1H) 7.23 (m, 5H), 4.33 (t, 0.1=7.2 Hz, 2H), 3.11 (t, J=7.6 Hz, 2H).

LCMS: 218.0 [M+H]⁺.

### Example 4: 3-Nitro-1-phenethyl-1H-pyrazole

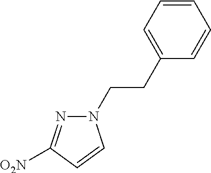 [SMILES: O=[N+]([O-])c1ccn(CCc2ccccc2)n1]

In the same manner as in Example 1, except that 3-nitro-1H-pyrazole was used instead of 4-aminopyridine, 0.1 g (12%) of a desired compound, which is a white solid, was obtained.

¹H NMR (400 MHz, DMSO-D6) δ 7.91 (d, J=2.4 Hz, 1H), 7.26 (m, 2H), 7.19 (m, 3H), 6.99 (d, J=2.0 Hz, 1H), 4.49 (t, 0.1=7.2 Hz, 2H), 3.15 (t, t, J=7.2 Hz, 2H).

LCMS: 218.0 [M+H]⁺.

### Example 5: 1-Phenethyl-1H-pyrazol-3-amine

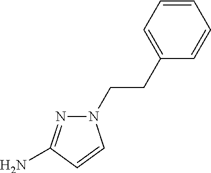 [SMILES: Nc1ccn(CCc2ccccc2)n1]

After dissolving the compound of Example 4 in methanol, Pd/C was added, and H₂ gas was added in the reactor. After stirring for 1 hour at room temperature, the mixture was filtered to remove Pd/C. The filtrate was concentrated under reduced pressure and dried under reduced pressure to obtain 0.1 g of a desired compound (12%).

¹H NMR (400 MHz, DMSO-D6) δ 7.28 (m, 2H), 7.15 (m, 4H), 5.30 (s, 1H), 4.52 (s, 2H), 4.03 (t, J=7.6 Hz, 2H), 2.99 (t, J=7.2 Hz, 2H).

LCMS: 188.2 [M+H]⁺.

### Example 6: 6-Amino-3-phenethylpyrimidin-4(3H)-one

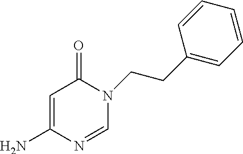 [SMILES: Nc1cc(=O)n(CCc2ccccc2)cn1]

In the same manner as in Example 1, except that 6-aminopyrimidin-4(3H)-one was used instead of 4-aminopyridine, 0.2 g (34%) of a desired compound, which is a white solid, was obtained.

¹H NMR (400 MHz, CD₃OD) δ 6.75 (m, 1H), 6.46 (m, 5H) 4.54 (s, 1H), 3.26 (t, J=7.2 Hz, 2H), 2.15 (t, J=7.2 Hz, 2H).

LCMS: 216.1 [M+H]⁺.

### Example 7: 4-Amino-2-bromo-1-phenethylpyridinium chloride

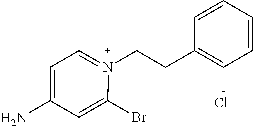

In the same manner as in Example 1, except that 2-bromopyridin-4-amine was used instead of 4-aminopyridine, 0.12 g (18.3%) of a desired compound, which is a white solid, was obtained.

¹H NMR (400 MHz, DMSO-D6) δ 8.49 (s, 2H), 8.16 (m, 1H), 7.32 (m, 2H), 7.3 (m, 1H), 7.22 (m, 2H), 7.03 (m, 1H), 6.79 (m, 1H), 4.50 (q, J=6.4 Hz, 2H), 3.09 (t, J=6.4 Hz, 2H).

LCMS: 278.9 [M].

### Example 8: 2,4-Diamino-1-phenethylpyridinium bromide

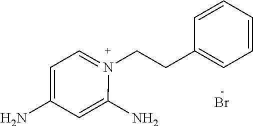

The compound of Example 7 was added to a sealed tube, and 30% of NH₄OH solution was added thereto. After stirring for 12 hours at 80° C., the mixture was cooled to room temperature. After concentration under reduced pressure, the mixture was dissolved in a small amount of methanol, and ethyl acetate was added to obtain a solid. The formed solid was filtered and dried under reduced pressure to obtain 39 mg (83%) of a desired compound, which is a white solid.

¹H NMR (400 MHz, DMSO-D6) δ 7.41 (s, 2H), 7.32 (m, 4H), 7.23 (m, 3H), 7.17 (m, 1H), 6.04 (d, J=7.6 Hz, 1H), 5.86 (s, 1H), 4.19 (t, J=6.8 Hz, 2H), 2.94 (t, J=6.8 Hz, 2H).

LCMS: 214.1 [M].

### Example 9: 1-Phenethyl-1H-imidazole

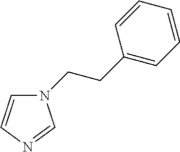 [SMILES: c1ccc(CCn2ccnc2)cc1]

In the same manner as in Example 1, except that 1H-imidazole was used instead of 4-aminopyridine, 0.35 g (27.7%) of a desired compound, which is a white solid, was obtained.

¹H NMR (400 MHz, DMSO-D6) δ 7.74 (s, 1H), 7.21 (d, J=7.6 Hz, 1H), 7.01 (d, J=7.6 Hz, 1H), 7.21 (m, 5H), 4.33 (t, J=7.2 Hz, 2H), 3.11 (t, J=7.6 Hz, 2H).

LCMS: 173.2 [M].

### Example 10: 2,6-Diamino-3-phenethylpyrimidin-4(3H)-one

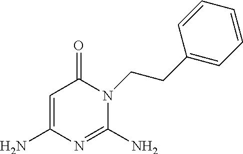 [SMILES: Nc1cc(=O)n(CCc2ccccc2)c(N)n1]

In the same manner as in Example 1, except that 2,6-diaminopyrimidin-4(3H)-one was used instead of 4-aminopyridine, 0.23 g (25.2%) of a desired compound, which is a white solid, was obtained.

¹H NMR (400 MHz, CD₃OD) δ 7.25 (m, 5H), 5.2 (s, 1H), 4.30 (t, J=7.2 Hz, 2H), 3.00 (t, J=7.2 Hz, 2H).

LCMS: 231.0 [M+H]⁺.

### Example 11: 4-Amino-1-(2-chlorophenethyl)pyridinium chloride

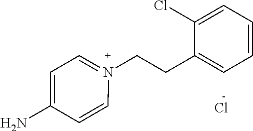

In the same manner as in Example 1, except that 1-chloro-2-(2-chloroethyl) benzene was used instead of (2-chloroethyl) benzene, 20 mg (5.85%) of a desired compound, which is a white solid, was obtained.

¹H NMR (400 MHz, DMSO-D6) δ 8.07 (s, 2H), 8.02 (d, J=6.8 Hz, 1H), 7.44 (m, 1H), 7.30 (m, 3H), 6.75 (d, J=7.2 Hz, 2H), 4.40 (t, J=6.4 Hz, 2H), 3.21 (t, J=6.4 Hz, 2H).

LCMS: 233.1, 235.1 [M].

### Example 12: 2,4-Diamino-1-(2-chlorophenethyl)pyridinium bromide

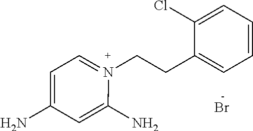

In the same manner as in Examples 7 and 8, except that 1-chloro-2-(2-chloroethyl) benzene was used instead of (2-chloroethyl) benzene, 21 mg (79%) of a desired compound, which is a white solid, was obtained.

¹H NMR (400 MHz, DMSO-D6) δ 7.48 (s, 2H), 7.43 (m, 1H), 7.34 (m, 2H), 7.29 (m, 2H), 7.11 (d, J=7.6 Hz, 2H), 6.02 (d, J=7.6 Hz, 1H), 5.88 (s, 1H), 4.23 (t, J=6.4 Hz, 2H), 3.01 (t, J=6.4 Hz, 2H).

LCMS: 248.1, 250.1 [M].

### Example 13: 3-Phenethylthiazol-3-ium iodide

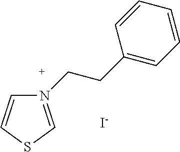

In the same manner as in Example 1, except that (2-iodoethyl) benzene was used instead of 4-aminopyridine, 40 mg (63.1%) of a desired compound, which is a white solid, was obtained.

¹H NMR (400 MHz, DMSO-D6) δ 10.03 (s, 1H), 8.57 (m, 1H), 8.23 (m, 1H), 7.23 (m, 5H), 4.82 (t, 0.1=7.2 Hz, 2H), 3.23 (t, J=7.2 Hz, 2H).

LCMS: 190.1 [M].

### Example 14: 2-Amino-3-phenethylthiazol-3-ium iodide

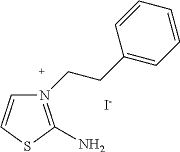

In the same manner as in Example 13, except that 2-amino thiazol was used instead of thiazol, 38 mg (58%) of a desired compound, which is a white solid, was obtained.

¹H NMR (400 MHz, DMSO-D6) δ 9.38 (s, 2H), 7.33 (m, 6H), 6.95 (s, 1H), 4.26 (t, J=7.6 Hz, 2H), 2.99 (t, J=7.2 Hz; 2H).

LCMS: 206.1 [M+H]⁺.

### Example 15: 4-Amino-2-cycloropyl-1-phenethylpyridinium iodide

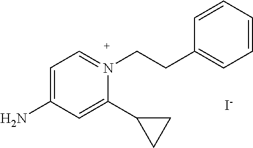

In the same manner as in Example 1, except that 2-cyclopropylpyridine-4-amine was used instead of 4-aminopyridine, 50 mg (12.3%) of a desired compound, which is a white solid, was obtained.

¹H NMR (400 MHz, DMSO-D6) δ 8.04 (d, J=6.8 Hz, 1H), 7.91 (s, 1H), 7.77 (s, 1H), 7.27 (m, 5H), 6.63 (m, 1H), 6.51 (m, 1H), 4.53 (t, J=7.6 Hz, 2H), 3.10 (t, J=7.2 Hz, 2H), 2.14 (m, 1H), 1.17 (m, 2H), 0.81 (m, 2H).

LCMS: 239.0 [M].

### Example 16: 4-Amino-1-phenethylquinolinium iodide

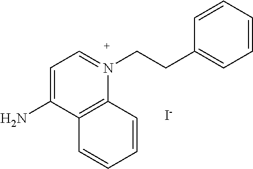

In the same manner as in Example 1, except that quinolin-4-amine was used instead of 4-aminopyridine, 60 mg (26.1%) of a desired compound, which is a white solid, was obtained.

¹H NMR (400 MHz, DMSO-D6) δ 8.9 (s, 1H), 8.47 (m, 1H), 8.25 (t, J=8.4 Hz, 2H), 8.05 (m, 1H) 7.70 (t, J=8.4 Hz, 1H), 7.26 (m, 3H), 7.22 (m, 2H), 6.67 (d, J=7.2 Hz, 1H), 4.80 (t, J=7.6 Hz, 2H), 3.13 (t, J=7.6 Hz, 2H).

LCMS: 249.0 [M].

### Example 17: 4-(Dimethylamino)-1-phenethylpyridinium chloride

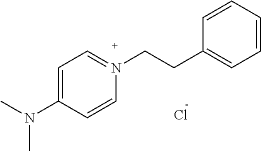

In the same manner as in Example 1, except that N,N-dimethylpyridin-4-amine was used instead of 4-aminopyridine, 50 mg (11.62%) of a desired compound, which is a white solid, was obtained.

¹H NMR (400 MHz, DMSO-D6) δ 8.26 (d, J=8.0 Hz, 2H), 7.32 (m, 2H), 7.23 (m, 3H), 7.00 (d, J=8.0 Hz, 2H), 4.44 (t, J=7.2 Hz, 2H), 3.13 (s, 6H), 3.11 (t. J=7.6 Hz, 2H).

LCMS: 227.1 [M].

### Example 18: 4-Amino-2-fluor-phenethylpyridinium chloride

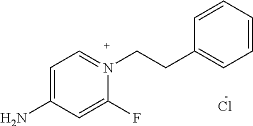

In the same manner as in Example 1, except that 2-fluororpyridin-4-amine was used instead of 4-aminopyridine, 0.1 g (22.18%) of a desired compound, which is a white solid, was obtained.

¹H NMR (400 MHz, DMSO-D6) δ 8.51 (m, 2H), 7.97 (t, J=6.4 Hz, 1H), 7.30 (m, 3H), 7.19 (m, 2H), 6.71 (d, J=7.6 Hz, 2H), 6.61 (d, J=7.6 Hz, 2H), 4.39 (t, J=6.8 Hz, 2H), 3.07 (t, J=6.8 Hz, 2H).

LCMS: 217.1 [M].

### Example 19: 4-Amino-1-(3,4-dichlorophenethyl)pyridinium chloride

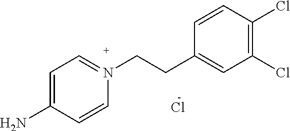

In the same manner as in Example 1, except that 1,2-dichloro-4-(2-chloroethyl) benzene was used instead of (2-chloroethyl) benzene, 0.1 g (15.5%) of a desired compound, which is a white solid, was obtained.

¹H NMR (400 MHz, DMSO-D6) δ 8.11 (s, 2H), 8.08 (d. J=7.8 Hz, 2H), 7.57 (d, J=7.8 Hz, 1H), 7.54 (s, 1H), 7.18 (d, J=7.8 Hz, 1H), 6.78 (d, J=7.8 Hz, 2H), 4.37 (t, J=7.8 Hz, 2H), 3.10 (t, J=7.8 Hz, 2H).

LCMS: 267.0, 269.0 [M].

### Example 20: 4-Amino-1-benzylpyridinium chloride

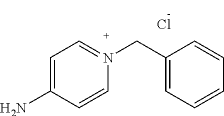

In the same manner as in Example 1, except that benzyl chloride was used instead of (2-chloroethyl) benzene, 0.3 g (42.6%) of a desired compound, which is a white solid, was obtained.

¹H NMR (400 MHz, DMSO-D6) S 8.41 (s, 2H), 8.31 (d, J=7.2 Hz, 2H), 7.39 (m, 5H), 6.89 (d, J=7.2 Hz, 2H), 5.37 (s, 2H).

LCMS: 185.1 [M].

### Example 21: 4-Amino-1-benzyl-2-fluoropyridinium chloride

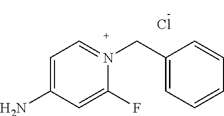

In the same manner as in Example 1, except that benzyl chloride was used instead of (2-chloroethyl) benzene and 2-fluoropyridine-4-amine was used instead of 4-amino pyridine, 0.2 g (47%) of a desired compound, which is a white solid, was obtained.

¹H NMR (400 MHz, CD₃OD) δ 8.09 (m, 1H), 7.43 (m, 3H), 7.40 (m, 2H), 6.80 (m, 1H), 6.63 (m, 1H), 5.37 (s, 2H).

LCMS: 203.1 [M].

### Example 22: 1-Phenethyl-5,6,7,8-tetrahydroquinolinium chloride

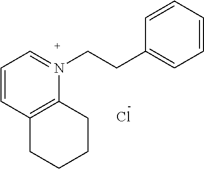

In the same manner as in Example 1, except that 5,6,7,8-tetrahydroquinolin-4-amine was used instead of 4-aminopyridine, 0.15 g (38.5%) of a desired compound, which is a white solid, was obtained.

¹H NMR (400 MHz, CD₃OD) δ 8.54 (m, 1H), 8.2 (m, 1H), 7.71 (m, 1H), 7.72 (m, 3H), 7.13 (m, 2H), 4.83 (m, 2H), 3.31 (m, 2H), 3.01 (m, 4H), 1.90 (m, 2H), 1.79 (m, 2H).

LCMS: 238.1 [M].

### Example 23: 4-Amino-1-(3-phenylpropyl)pyridinium chloride

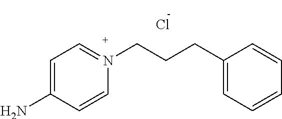

In the same manner as in Example 1, except that (3-chloropropyl) benzene was used instead of (2-chloroethyl) benzene, 0.1 g (38%) of a desired compound, which is a white solid, was obtained.

¹H NMR (400 MHz, DMSO-D6) δ 8.26 (s, 2H), 8.20 (d, J=7.2 Hz, 2H), 7.28 (m, 2H), 7.21 (m, 3H), 6.86 (d, J=7.2 Hz, 2H), 4.15 (t, J=7.2 Hz, 2H), 2.57 (m, 2H), 2.08 (m, 2H).

LCMS: 213.0 [M].

### Example 24: 4-Amino-2-fluoro-1-(3-phenylpropyl)pyridinium chloride

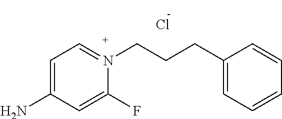

In the same manner as in Example 1, except that (3-chloropropyl) benzene was used instead of (2-chloroethyl) benzene and 2-fluoropyridine-4-amine was used instead of 4-aminopyridine, 55 mg (15.41%) of a desired compound, which is a white solid, was obtained.

¹H NMR (400 MHz, CD₃OD) δ 7.94 (m, 1H), 7.21 (m, 5H), 6.72 (m, 1H), 6.54 (m, 1H), 4.21 (m, 2H), 2.73 (m, 2H), 2.16 (m, 2H).

LCMS: 231.1 [M].

### Example 25: 4-Amino-1-(2-oxo-2-(4-(trifluoromethoxy)phenyl)ethyl)pyridinium bromide

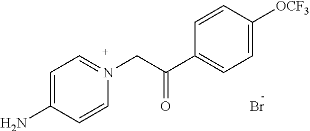

In the same manner as in Example 1, except that 2-bromo-1-(4-(trifluoromethoxy)phenyl)ethanone was used instead of (2-chloroethyl) benzene, 45 mg (53%) of a desired compound, which is a white solid, was obtained.

¹H NMR (400 MHz, DMSO-D6) S 8.27 (s, 2H), 8.16 (m, 2H), 8.08 (d, J=7.2 Hz, 2H), 7.64 (m, 2H), 6.92 (d, J=7.6 Hz, 2H), 5.97 (s, 2H).

LCMS: 297.0 [M].

### Example 26: 4-Amino-1-(2-oxo-2-phenylethyl)pyridinium bromide

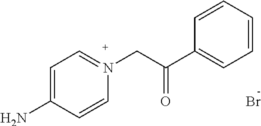

In the same manner as in Example 1, except that 2-bromo-1-(phenyl)ethanone was used instead of (2-chloroethyl) benzene, 45 mg (53%) of a desired compound, which is a white solid, was obtained.

¹H NMR (400 MHz, DMSO-D6) δ 7.99 (m, 4H), 7.66 (m, 1H), 7.53 (m 2H), 6.81 (m, 2H), 4.34 (s, 2H).

LCMS: 213.1 [M].

### Example 27: 4-Amino-1-(2-cyclohexylethyl)pyridinium bromide

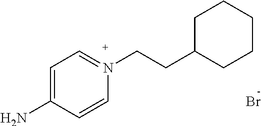

In the same manner as in Example 1, except that (2-bromoethyl)cyclohexane was used instead of (2-chloroethyl) benzene, 0.15 g (24.75%) of a desired compound, which is a white solid, was obtained.

¹H NMR (400 MHz, DMSO-D6) δ 8.10 (d. J=6.4 Hz, 2H), 6.84 (d, J=6.4 Hz, 2H), 4.17 (m, 2H), 1.77 (m, 7H), 1.24 (m, 4H), 1.00 (m, 2H).

LCMS: 205.2 [M].

### Example 28: 4-Amino-1-(2-cyclohexylethyl)-2-fluoropyridinium bromide

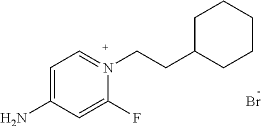

In the same manner as in Example 1, except that (2-bromoethyl)cyclohexane was used instead of (2-chloroethyl) benzene and 2-fluoropyridine-4-amine was used instead of 4-aminopyridine, 0.35 g (64.7%) of a desired compound, which is a white solid, was obtained.

¹H NMR (400 MHz, DMSO-D6) δ 8.00 (m, 1H), 6.76 (m, 1H), 6.60 (m, 1H), 4.20 (m, 2H), 1.76 (m, 7H), 1.22 (m, 4H), 0.98 (m, 2H).

LCMS: 223.1 [M].

### Example 29: 2,4-Diamino-1-benzylpyridinium chloride

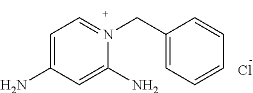

In the same manner as in Example 1, except that benzyl chloride was used instead of (2-chloroethyl) benzene and pyridine-2,4-diamine was used instead of 4-aminopyridine, 0.2 g (46.3%) of a desired compound, which is a white solid, was obtained.

¹H NMR (400 MHz, DMSO-D6) δ 7.72 (d, J=7.2 Hz, 1H), 7.45 (s, 2H), 7.34 (m, 5H), 7.20 (m, 2H), 6.24 (m, 1H), 5.91 (m, 1H), 5.24 (s, 2H).

LCMS: 200.1 [M].

### Example 30: 4-Amino-1-benzyl-2-chloropyridinium chloride

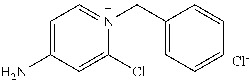

In the same manner as in Example 1, except that benzyl chloride was used instead of (2-chloroethyl) benzene and 2-chloropyridin-4-amine was used instead of 4-aminopyridine, 0.15 g (37.8%) of a desired compound, which is a white solid, was obtained.

¹H NMR (400 MHz, DMSO-D6) δ 8.34 (d. J=13.6 Hz, 2H), 8.50 (d, J=7.2 Hz, 1H), 7.36 (m, 4H), 7.25 (m, 2H), 7.14 (m, 1H), 6.97 (m, 1H).

LCMS: 219.1 [M].

### Example 31: 4-Amino-1-(cyclopropylmethyl)pyridinium chloride

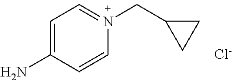

In the same manner as in Example 1, except that (chloromethyl)cyclopropane was used instead of (2-chloroethyl) benzene, 0.18 g (30.6%) of a desired compound, which is a white solid, was obtained.

¹H NMR (400 MHz, DMSO-D6) δ 8.22 (d, J=7.2 Hz, 2H), 8.19 (s, 2H), 6.86 (d, J=7.2 Hz, 2H), 4.00 (d. J=7.6 Hz, 2H), 1.24 (m, 1H), 0.59 (m, 2H), 0.43 (m, 2H).

LCMS: 149.2 [M].

### Example 32: 4-Amino-2-chloro-1-phenethylpyridinium chloride

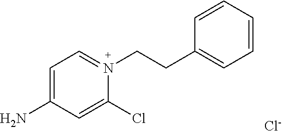

In the same manner as in Example 30, except that (2-chloroethyl) benzene was used instead of benzyl chloride, 0.08 g (25.5%) of a desired compound, which is a white solid, was obtained.

¹H NMR (400 MHz, CD₃OD) δ 7.89 (d, J=7.6 Hz, 1H), 7.42 (m, 3H), 7.24 (m, 2H), 7.00 (m, 1H), 6.70 (m, 1H), 4.57 (t, J=7.2 Hz, 2H), 3.16 (t. J=7.6 Hz, 2H).

LCMS: 233.1, 235.1 [M, M+2]⁺.

### Example 33: 4-(Methylamino)-1-phenethylpyridinium chloride

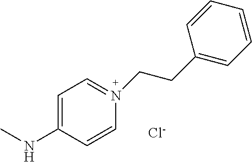

In the same manner as in Example 1, except that N-methylpyridin-4-amine was used instead of 4-aminopyridine, 0.12 g (26.1%) of a desired compound, which is a white solid, was obtained.

¹H NMR (400 MHz, DMSO-D6) δ 9.04 (m, 1H), 8.23 (m, 1H), 8.05 (m, 1H), 7.32 (m, 2H), 7.23 (m, 3H), 6.91 (m, 1H), 6.80 (m, 1H), 4.31 (t, J=7.2 Hz, 2H), 3.09 (t. J=7.2 Hz, 2H), 2.86 (d, J=4.8 Hz, 3H).

LCMS: 213.1 [M].

### Example 34: 1-Benzyl-4-(methylamino)pyridinium chloride

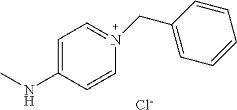

In the same manner as in Example 1, except that N-methylpyridin-4-amine was used instead of 4-aminopyridine and benzyl chloride was used instead of (2-chloroethyl) benzene, 0.2 g (46.1%) of a desired compound, which is a white solid, was obtained.

¹H NMR (400 MHz, DMSO-D6) δ 9.19 (m, 1H), 8.49 (d, J=7.2 Hz, 1H), 8.27 (d, J=7.2 Hz, 1H), 7.38 (m, 5H), 6.99 (m, 1H), 6.88 (m, 1H), 5.39 (s, 2H), 2.88 (d, J=6.0 Hz, 3H).

LCMS: 199.1 [M].

### Example 35: 4-Amino-1-(3,4-dichlorobenzyl)-2-fluoropyridinium chloride

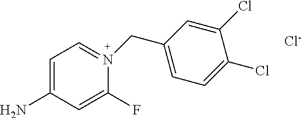

In the same manner as in Example 1, except that 3,4-dichlorobenzyl chloride was used instead of (2-chloroethyl) benzene and 4-amino-2-fluoropyridine was used instead of 4-aminopyridine, 50 mg (12.9%) of a desired compound, which is a white solid, was obtained.

¹H NMR (400 MHz, DMSO-D6) δ 8.79 (m, 2H), 8.26 (t, J=6.4 Hz, 1H), 7.71 (m, 2H), 7.34 (m, 1H), 6.86 (m, 1H), 6.73 (m, 1H), 5.40 (s, 2H).

LCMS: 271.0, 273.0 [M. M+2]⁺.

### Example 36: 4-Amino-1-(3,4-dichlorobenzyl)pyridinium chloride

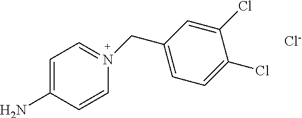

In the same manner as in Example 1, except that 3,4-dichlorobenzyl chloride was used instead of (2-chloroethyl) benzene, 0.11 g (23.8%) of a desired compound, which is a white solid, was obtained.

¹H NMR (400 MHz, DMSO-D6) δ 8.50 (s, 2H), 8.36 (d, J=6.8 Hz, 2H), 7.80 (m, 1H), 7.71 (d, J=8.0 Hz, 1H), 7.42 (m, 1H), 6.93 (d, J=7.6 Hz, 2H), 5.41 (s, 2H).

LCMS: 253.0, 255.0 [M, M+2]⁺.

### Example 37: 1-(3,4-Dichlorobenzyl)-4-(methylamino)pyridinium chloride

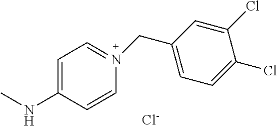

In the same manner as in Example 1, except that N-methylpyridin-4-amine was used instead of 4-aminopyridine and 3,4-dichlorobenzyl chloride was used instead of (2-chloroethyl) benzene, 0.15 g (26.7%) of a desired compound, which is a white solid, was obtained.

¹H NMR (400 MHz, DMSO-D6) δ 9.22 (m, 1H), 8.50 (m, 1H), 8.28 (m, 1H), 7.80 (m, 1H), 7.1 (d, J=8.0 Hz, 1H), 7.43 (m, 1H), 7.00 (m, 1H), 6.89 (m, 1H), 5.40 (s, 2H), 2.51 (d, J=5.2 Hz, 3H).

LCMS: 267.0, 269.0 [M, M+2].

### Example 38: 1-(3,4-Dichlorobenzyl)-4-(dimethylamino)pyridinium chloride

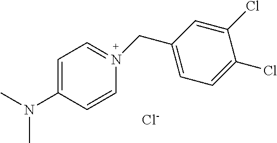

In the same manner as in Example 1, except that 3,4-dimethylpyridin-4-amine was used instead of 4-aminopyridine and 3,4-dichlorobenzyl chloride was used instead of (2-chloroethyl) benzene, 0.12 g (23.08%) of a desired compound, which is a white solid, was obtained.

¹H NMR (400 MHz, DMSO-D6) δ 8.51 (d, J=8.0 Hz, 2H), 7.83 (m, 1H), 7.71 (d, J=8.4 Hz, 1H), 7.45 (m, 1H), 7.08 (d, J=7.6 Hz, 2H), 5.45 (s, 2H), 3.15 (s, 6H).

LCMS: 281.0, 283.0 [M, M+2]⁺.

### Example 39: 4-Amino-1-(cyclopropylmethyl)-2-fluoropyridinium chloride

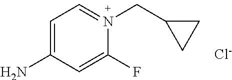

In the same manner as in Example 1, except that 4-amino-2-fluoropyridine was used instead of 4-aminopyridine and (2-chloromethyl)cyclopropane was used instead of (2-chloroethyl) benzene, 0.07 g (25.8%) of a desired compound, which is a white solid, was obtained.

¹H NMR (400 MHz, DMSO-D6) δ 8.82 (s, 2H), 8.22 (m, 1H), 6.89 (m, 1H), 6.80 (m, 1H), 4.01 (m, 2H), 1.23 (m, 1H) 0.60 (m, 2H), 0.44 (m, 2H).

LCMS: 167.1 [M].

### Example 40: 2,4-Diamino-1-(2-cyclohexylethyl)pyridinium bromide

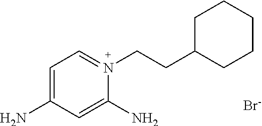

In the same manner as in Examples 7 and 8, except that (2-chloroethyl) cyclohexane was used instead of (2-chloroethyl) benzene, 30 mg (19.96%) of a desired compound, which is a white solid, was obtained.

¹H NMR (400 MHz, DMSO-D6) δ 7.72 (d, J=7.2 Hz, 1H), 7.45 (s, 2H), 7.20 (m, 1H), 6.24 (m, 1H), 4.17 (m, 2H), 1.77 (m, 7H), 1.24 (m, 4H), 1.00 (m, 2H).

LCMS: 220.1 [M].

### Example 41: 1-(Cyclopropylmethyl)-4-(methylamino)pyridinium chloride

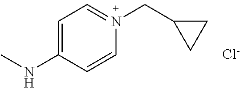

In the same manner as in Example 34, except that (2-chloromethyl)cyclopropane was used instead of benzyl chloride, 0.09 g (24.4%) of a desired compound, which is a white solid, was obtained.

¹H NMR (400 MHz, DMSO-D6) δ 9.22 (m, 1H), 8.42 (m, 1H), 8.22 (m, 1H), 7.03 (m, 1H), 6.83 (m, 1H) 4.08 (d, J=7.2 Hz, 2H), 2.88 (d. J=5.2 Hz, 3H), 1.28 (m, 1H), 0.56 (m, 2H), 0.46 (m, 2H).

LCMS: 163.2 [M].

### Example 42: 1-(Cyclopropylmethyl)-4-(dimethylamino)pyridinium chloride

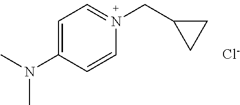

In the same manner as in Example 1, except that N,N-dimethylpyridin-4-amine was used instead of 4-aminopyridine and (2-chloromethyl) cyclopropane was used instead of (2-chloroethyl) benzene, 90 mg (24%) of a desired compound, which is a white solid, was obtained.

¹H NMR (400 MHz, DMSO-D6) δ 8.42 (d, J=7.6 Hz, 2H), 7.07 (d, J=7.6 Hz, 2H), 4.09 (d, J=7.6 Hz, 2H), 3.13 (s, 6H), 1.28 (m, 1H), 0.56 (m, 2H), 0.48 (m, 2H).

LCMS: 177.2 [M].

### Example 43: 4-Amino-3-methyl-1-benzylpyridinium chloride

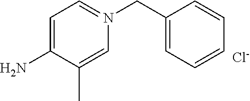

In the same manner as in Example 1, except that 3-methylpyridin-4-amine was used instead of 4-aminopyridine and benzyl chloride was used instead of (2-chloroethyl) benzene, 0.12 g (27.6%) of a desired compound, which is a white solid, was obtained.

¹H NMR (400 MHz, DMSO-D6) δ 8.62 (s, 1H), 8.37 (s, 1H), 8.29 (m, 1H), 7.71 (s, 1H), 7.38 (m, 5H), 6.95 (d, J=6.8 Hz, 1H), 5.36 (s, 2H), 2.09 (s, 3H).

LCMS: 199.1 [M].

### Example 44: 4-Amino-3-methyl-1-phenethylpyridinium chloride

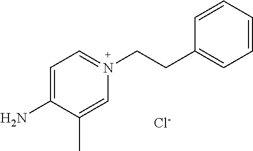

In the same manner as in Example 1, except that 3-methylpyridin-4-amine was used instead of 4-aminopyridine, 0.09 g (19.5%) of a desired compound, which is a white solid, was obtained.

¹H NMR (400 MHz, DMSO-D6) δ 8.61 (s, 1H), 8.22 (s, 1H), 8.08 (m, 1H), 7.31 (m, 5H), 6.89 (d, J=7.2 Hz, 1H), 4.36 (t, J=7.2 Hz, 2H), 3.10 (t, J=6.8 Hz, 2H), 2.09 (s, 3H).

LCMS: 213.1 [M].

### Example 45: 4-Amino-1-benzyl-2-methoxypyridinium chloride

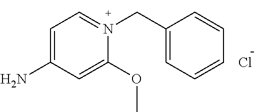

In the same manner as in Example 1, except that 2-methoxylpyridin-4-amine was used instead of 4-aminopyridine and benzyl chloride was used instead of (2-chloroethyl) benzene, 0.08 g (26.4%) of a desired compound, which is a white solid, was obtained.

¹H NMR (400 MHz, DMSO-D6) δ 8.21 (s, 1H), 8.15 (d, J=7.2 Hz, 1H), 7.38 (m, 6H), 6.62 (m, 1H), 6.36 (m, 1H), 5.25 (s, 2H), 4.01 (s, 3H).

LCMS: 215.1 [M].

### Example 46: 4-Amino-1-(cyclohexylmethyl)pyridinium bromide

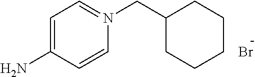

In the same manner as in Example 1, except that (2-chloromethyl)cyclohexane was used instead of (2-chloroethyl) benzene, 0.1 g (23.1%) of a desired compound, which is a white solid, was obtained.

¹H NMR (400 MHz; DMSO-D6) δ 8.19 (s, 2H), 8.17 (d, 0.1=5.6 Hz, 2H), 6.87 (d, J=1=6.4 Hz, 2H), 4.00 (d, J=7.6 Hz, 2H), 1.7 (m, 6H), 0.99 (m, 5H).

LCMS: 191.2 [M].

### Example 47: 4-Amino-1-(cyclobutylmethyl)pyridinium bromide

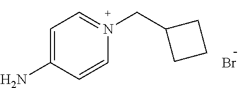

In the same manner as in Example 1, except that (2-chloromethyl)cyclobutane was used instead of (2-chloroethyl) benzene, 0.045 g (35.4%) of a desired compound, which is a white solid, was obtained.

¹H NMR (400 MHz, DMSO-D6) 8.19 (m, 2H), 8.09 (s, 2H), 6.84 (m, 2H), 4.16 (m, 2H), 2.69 (m, 1H), 1.83 (m, 6H).

LCMS: 163.2 [M].

### Example 48: 4-Amino-1-(cyclobutylmethyl)-2-fluoropyridinium bromide

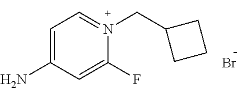

In the same manner as in Example 1, except that 4-amino-2-fluoropyridine was used instead of 4-aminopyridine and (2-chloromethyl)butane was used instead of (2-chloroethyl) benzene, 0.003 g (31.7%) of a desired compound, which is a white solid, was obtained.

¹H NMR (400 MHz, DMSO-D6) δ 8.02 (m, 2H), 6.79 (m, 1H), 6.62 (m, 1H), 4.20 (m, 2H), 2.81 (m, 1H), 1.93 (m, 6H).

LCMS: 181.2 [M].

### Example 49: 4-Amino-1-(4-fluorobenzyl)pyridinium bromide

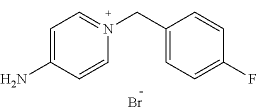

In the same manner as in Example 1, except that 4-fluorobenzyl bromide was used instead of (2-chloroethyl) benzene, 0.078 g (45.8%) of a desired compound, which is a white solid, was obtained.

¹H NMR (400 MHz, DMSO-D6) δ 8.36 (m, 2H), 8.24 (s, 2H), 7.52 (m, 2H), 7.27 (m, 2H), 6.88 (m, 2H), 5.41 (s, 2H).

LCMS: 203.1 [M].

### Example 50: 1-Benzyl-4-morpholinopyridinium chloride

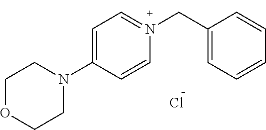

In the same manner as in Example 1, except that benzyl chloride was used instead of (2-chloroethyl) benzene and 4-(pyridine-4-yl)morpholine was used instead of 4-aminopyridine, 0.078 g (22%) of a desired compound, which is a white solid, was obtained.

¹H NMR (400 MHz, DMSO-D6) δ 8.55 (m, 2H), 7.45 (m, 5H), 7.38 (m, 2H), 5.45 (s, 2H), 3.71 (m, 8H).

LCMS: 255.1 [M].

### Example 51: 4-Morpholino-1-phenethylpyridinium chloride

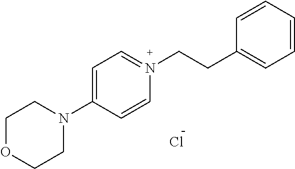

In the same manner as in Example 1, except that 4-(pyridine-4-yl)morpholine was used instead of 4-aminopyridine, 0.087 g (23.4%) of a desired compound, which is a white solid, was obtained.

¹H NMR (400) MHz, DMSO-D6) δ 8.33 (m, 2H), 7.32 (m, 2H), 7.22 (m, 5H), 4.47 (m, 2H), 3.67 (m, 8H), 3.10 (m, 2H).

LCMS: 269.1 [M].

### Example 52: 4-Morpholino-1-(cyclopropylmethyl)pyridinium chloride

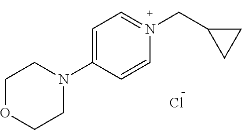

In the same manner as in Example 1, except that 4-(pyridine-4-yl)morpholine was used instead of 4-aminopyridine and (2-chloromethyl)cyclopropane was used instead of (2-chloroethyl) benzene, 0.069 g (22.2%) of a desired compound, which is a white solid, was obtained.

¹H NMR (400 MHz, DMSO-D6) δ 8.58 (m, 2H), 7.58 (m, 2H), 4.09 (d, J=7.6 Hz, 2H), 3.71 (m, 8H), 1.28 (m, 1H), 0.56 (m, 2H), 0.48 (m, 2H).

LCMS: 219.1 [M].

### Example 53: 1-(2-Cyclohexylethyl)-4-morpholinopyridinium bromide

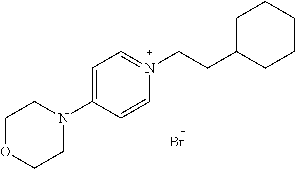

In the same manner as in Example 1, except that 4-(pyridine-4-yl)morpholine was used instead of 4-aminopyridine and (2-chloroethyl)cyclohexane was used instead of (2-chloroethyl) benzene, 0.101 g (23.3%) of a desired compound, which is a white solid, was obtained.

¹H NMR (400 MHz, DMSO-D6) δ 8.58 (m, 2H), 7.58 (m, 2H), 4.17 (m, 2H), 3.71 (m, 8H), 1.77 (m, 7H), 1.24 (m, 4H), 1.00 (m, 2H).

LCMS: 275.1 [M].

### Example 54: 1-Benzyl-4-(pyrrolidin-1-yl)pyridinium chloride

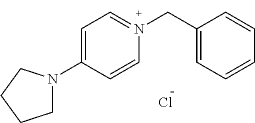

In the same manner as in Example 1, except that 4-(pyrrolidin-1-yl)pyridine was used instead of 4-aminopyridine and benzyl chloride was used instead of (2-chloroethyl) benzene, 0.25 g (22.2%) of a desired compound, which is a white solid, was obtained.

¹H NMR (400 MHz, DMSO-D6) δ 8.44 (d, J=7.6 Hz, 2H), 7.38 (m, 5H), 6.92 (d, J=7.6 Hz, 2H), 5.41 (s, 2H), 3.50 (m, 4H), 2.01 (m, 4H).

LCMS: 239.3 [M].

### Example 55: 1-Phenethyl-4-(pyrrolidin-1-yl)pyridinium chloride

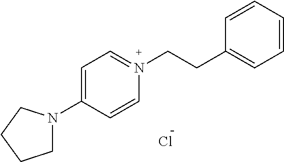

In the same manner as in Example 1, except that 4-(pyrrolidin-1-yl)pyridine was used instead of 4-aminopyridine, 0.15 g (30.8%) of a desired compound, which is a white solid, was obtained.

¹H NMR (400 MHz. DMSO-D6) δ 8.26 (d, J=8 Hz, 2H), 7.24 (m, 5H), 6.86 (d, J=8 Hz, 2H), 4.46 (t. J=7.6 Hz, 2H), 3.48 (m, 4H), 3.13 (t, J=7.6 Hz, 2H), 1.99 (m, 4H).

LCMS: 253.3 [M].

### Example 56: 1-(Cyclopropylmethyl)-4-(pyrrolidin-1-yl)pyridinium chloride

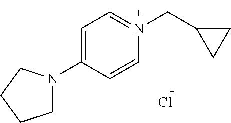

In the same manner as in Example 1, except that 4-(pyrrolidin-1-yl)pyridine was used instead of 4-aminopyridine and (2-chloromethyl)cyclopropane was used instead of (2-chloroethyl) benzene, 0.2 g (49.7%) of a desired compound, which is a white solid, was obtained.

¹H NMR (400 MHz, DMSO-D6) δ 8.40 (d. J=8 Hz, 2H), 6.92 (d. J=8 Hz, 2H), 4.07 (d, J=7.2 Hz, 2H), 3.51 (m, 4H), 2.01 (m, 4H), 1.29 (m, 1H), 0.47 (m, 4H).

LCMS: 203.1 [M].

### Example 57: 1-(Cyclohexylmethyl)-4-(pyrrolidin-1-yl)pyridinium bromide

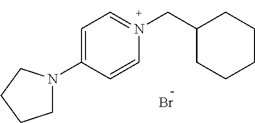

In the same manner as in Example 1, except that 4-(pyrrolidin-1-yl)pyridine was used instead of 4-aminopyridine and (2-chloromethyl)cyclohexane was used instead of (2-chloroethyl) benzene, 0.21 g (47.8%) of a desired compound, which is a white solid, was obtained.

¹H NMR (400 MHz, DMSO-D6) δ 8.29 (d, J=7.6 Hz, 2H), 6.91 (d, J=7.6 Hz, 2H), 4.05 (d, J=7.2 Hz, 2H), 3.51 (m, 4H), 2.01 (m, 4H), 1.70 (m, 4H), 1.49 (m, 2H), 1.15 (m, 3H), 0.97 (m, 2H).

LCMS: 245.3 [M].

### Example 58: 1-(Cyclobutylmethyl)-4-(pyrrolidin-1-yl)pyridinium chloride

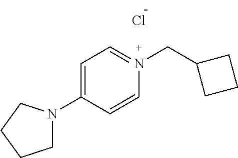

In the same manner as in Example 1, except that 4-(pyrrolidin-1-yl)pyridine was used instead of 4-aminopyridine and (2-chloromethyl)cyclobutane was used instead of (2-chloroethyl) benzene, 0.13 g (38.1%) of a desired compound, which is a white solid, was obtained.

¹H NMR (400 MHz, DMSO-D6) δ 8.29 (d, J=7.6 Hz, 2H), 6.89 (d, 0.7.6 Hz, 2H), 4.20 (d, J=7.2 Hz, 2H), 3.48 (m, 4H), 2.72 (m, 1H), 2.01 (m, 4H), 1.84 (m, 6H),

LCMS: 217.2 [M].

### Example 59: 1-Benzyl-4-(piperidin-1-yl)pyridinium chloride

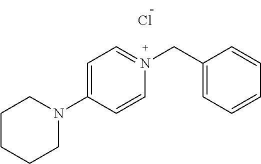

In the same manner as in Example 1, except that 4-(piperidin-1-yl)pyridine was used instead of 4-aminopyridine and benzyl chloride was used instead of (2-chloroethyl) benzene, 0.078 g (32.4%) of a desired compound, which is a white solid, was obtained.

¹H NMR (400 MHz, DMSO-D6) δ 8.38 (d, J=7.6 Hz, 2H), 7.42 (m, 5H), 7.24 (d, J=7.6 Hz, 2H), 5.35 (s, 2H), 3.67 (m, 4H), 1.65 (m, 2H), 1.59 (m, 4H).

LCMS: 253.2 [M].

### Example 60: 4-(Azepan-1-yl)-1-benzylpyridinium chloride

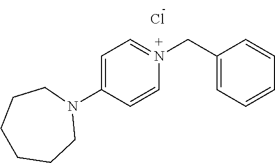

In the same manner as in Example 1, except that 4-(azepan-1-yl)pyridine was used instead of 4-aminopyridine and benzyl chloride was used instead of (2-chloroethyl) benzene, 0.081 g (32.1%) of a desired compound, which is a white solid, was obtained.

¹H NMR (400 MHz, DMSO-D6) δ 8.39 (d, J=7.2 Hz, 2H), 7.42 (m, 5H), 7.13 (d, J=7.2 Hz, 2H), 5.38 (s, 2H), 3.69 (m, 4H), 1.72 (m, 4H), 1.47 (m, 4H).

LCMS: 267.2 [M].

### Example 61: 1-Benzyl-4-(neopentylamino)pyridinium chloride

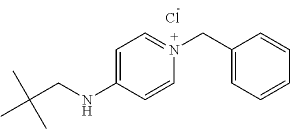

In the same manner as in Example 1, except that 4-(neopentylamino)pyridine was used instead of 4-aminopyridine and benzyl chloride was used instead of (2-chloroethyl) benzene, 0.085 g (35.1%) of a desired compound, which is a white solid, was obtained.

¹H NMR (400 MHz, DMSO-D6) δ 8.69 (t, J=6.4 Hz, 2H), 8.40 (d, J=7.6 Hz, 1H), 8.22 (d, J=7.6 Hz, 1H), 7.38 (m, 5H), 7.06 (m, 2H), 5.34 (s, 2H), 3.12 (d, J=6.4 Hz, 2H), 0.93 (s, 9H).

LCMS: 255.2 [M].

### Example 62: 4-(Pyrrolidin-1-yl)-1-(thiophen-3-ylmethyl)pyridinium bromide

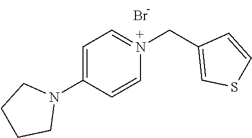

In the same manner as in Example 1, except that 4-(pyrrolidin-1-yl)pyridine was used instead of 4-aminopyridine and 3-(bromomethyl)thiophene was used instead of (2-chloroethyl) benzene, 0.029 g (67.2%) of a desired compound, which is a white solid, was obtained.

¹H NMR (400 MHz, DMSO-D6) δ 8.39 (d, J=7.6 Hz, 2H), 7.61 (m, 2H), 7.16 (m, 1H), 6.91 (d, J=7.6 Hz, 2H), 5.38 (s, 2H), 3.46 (m, 4H), 1.99 (m, 4H).

LCMS: 245.1 [M].

### Example 63: 6-(Cyclopropylmethyl)-1,2,3,4-tetrahydro-1,6-naphthyridin-6-ium chloride

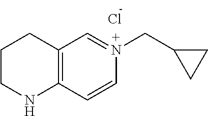

In the same manner as in Example 1, except that 1,2,3,4-tetrahydro-1,6-naphthyridine was used instead of 4-aminopyridine and (2-chloromethyl)cyclopropane was used instead of (2-chloroethyl) benzene, 0.039 g (23%) of a desired compound, which is a white solid, was obtained.

¹H NMR (400 MHz, DMSO-D6) δ 9.24 (s, 1H), 8.16 (s, 1H), 8.10 (m, 1H), 6.83 (d, J=7.2 Hz, 1H), 3.94 (d, J=7.6 Hz, 2H), 3.35 (m, 2H), 2.69 (m, 2H), 1.78 (m, 2H), 1.24 (m, 1H), 0.52 (m, 2H), 0.44 (m, 2H).

LCMS: 189.2 [M].

### Example 64: 6-(Cyclopropylmethyl)-2,3-dihydro-1H-pyrido[3,4-b][1,4]oxazin-6-ium chloride

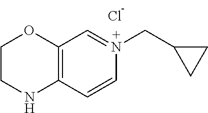

In the same manner as in Example 1, except that 1,2,3,4-tetrahydro-1,6-naphthyridine was used instead of 4-aminopyridine and (2-chloromethyl)cyclopropane was used instead of (2-chloroethyl) benzene, 0.019 g (23%) of a desired compound, which is a white solid, was obtained.

¹H NMR (400 MHz, CD₃OD) δ 8.07 (d, J=2 Hz, 2H), 7.98 (d, J=7.2 Hz, 2H), 6.87 (d, J=7.2 Hz, 2H), 4.31 (t, J=4.8 Hz, 2H), 4.12 (d, J=6.8 Hz, 2H), 3.64 (t, J=4.8 Hz, 2H), 1.35 (m, 1H), 0.72 (m, 2H), 0.53 (m, 2H).

LCMS: 191.2 [M].

### Example 65: l-Benzyl-4(4,4-difluoropiperidin-1-yl)pyridinium chloride

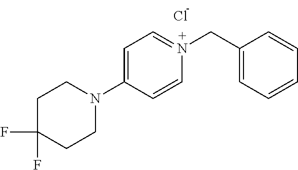

In the same manner as in Example 1, except that 4-(4,4-difluoropiperidin-1-yl)pyridine was used instead of 4-aminopyridine and benzyl chloride was used instead of (2-chloroethyl) benzene, 0.018 g (16%) of a desired compound, which is a white solid, was obtained.

¹H NMR (400 MHz. CD₃OD) δ 8.30 (d, J=7.6 Hz, 2H), 7.41 (m, 5H), 7.40 (d, J=7.6 Hz, 2H), 5.38 (s, 2H), 3.86 (m, 4H), 2.17 (m, 4H).

LCMS: 289.2 [M].

### Example 66: 4-(Azetidin-1-yl)-1-benzylpyridinium chloride

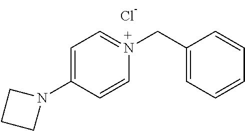

In the same manner as in Example 1, except that 4-(azetidin-1-yl)pyridine was used instead of 4-aminopyridine and benzyl chloride was used instead of (2-chloroethyl) benzene, 0.016 g (18%) of a desired compound, which is a white solid, was obtained.

¹H NMR (400 MHz, CD₃OD) δ 8.18 (d, J=7.2 Hz, 2H), 7.42 (m, 5H), 6.63 (d, J=7.2 Hz, 2H), 5.33 (s, 2H), 4.31 (t, J=8 Hz, 4H), 2.56 (m, 2H).

LCMS: 225.2 [M].

### Example 67: 1-Benzyl-4-(oxetan-3-ylamino)pyridinium chloride

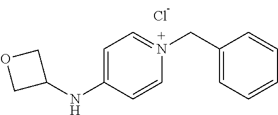

In the same manner as in Example 1, except that 4-(oxetan-3-ylamino)pyridine was used instead of 4-aminopyridine and benzyl chloride was used instead of (2-chloroethyl) benzene, 0.012 g (13%) of a desired compound, which is a white solid, was obtained.

¹H NMR (400 MHz, DMSO-D6) δ 9.45 (m, 1H), 8.48 (d, J=7.6 Hz, 1H), 8.31 (d, 0.1=7.6 Hz, 1H), 7.39 (m, 5H), 6.96 (m, 1H), 6.83 (m, 1H), 5.39 (s, 2H), 4.87 (s, 2H), 4.50 (s, 2H).

LCMS: 241.2 [M].

### Example 68: 4-(Pyrrolidin-1-yl)-1-(thiophen-2-ylmethyl)pyridinium chloride

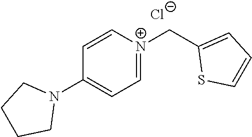 [SMILES: [Cl-] ; c1csc(C[n+]2ccc(N3CCCC3)cc2)c1]

In the same manner as in Example 1, except that 4-(pyrrolidin-1-yl)pyridine was used instead of 4-aminopyridine and 2-(bromomethyl)thiophene was used instead of (2-chloroethyl) benzene, 0.018 g (14.6%) of a desired compound, which is a white solid, was obtained.

¹H NMR (400 MHz, DMSO-D6) δ 8.41 (d, J=7.2 Hz, 2H), 7.60 (m, 1H), 7.31 (d, J=3.6 Hz, 2H), 7.07 (m, 1H), 6.91 (d, J=7.2 Hz, 2H), 5.61 (s, 2H), 3.49 (m, 4H), 1.98 (m, 4H),

LCMS: 245.1 [M].

### Example 69: l-Benzyl-4-(tert-butylamino)pyridinium chloride

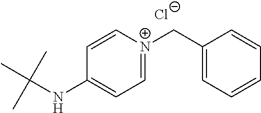 [SMILES: [Cl-] ; CC(C)(C)Nc1cc[n+](Cc2ccccc2)cc1]

In the same manner as in Example 1, except that 4-(tert-butylamino)pyridine was used instead of 4-aminopyridine and benzyl chloride was used instead of (2-chloroethyl)benzene, 0.021 g (12%) of a desired compound, which is a white solid, was obtained.

¹H NMR (400 MHz, DMSO-D6) δ 9.71 (s, 1H), 8.09 (d, J=6.8 Hz, 1H), 7.97 (m, 1H), 7.84 (d, J=6.8 Hz, 1H), 7.41 (m, 3H), 7.36 (m, 2H), 6.72 (m, 1H), 5.37 (s, 2H), 1.47 (s, 9H),

LCMS: 241.1 [M].

### Example 70: 4-(Azetidin-1-yl)-1-(cyclopropylmethyl)pyridinium chloride

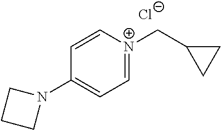 [SMILES: [Cl-] ; c1c[n+](CC2CC2)ccc1N1CCC1]

In the same manner as in Example 1, except that 4-(azetidin-1-yl)pyridine was used instead of 4-aminopyridine and (2-chloromethyl)cyclopropane was used instead of (2-chloroethyl)benzene, 0.045 g (53.7%) of a desired compound, which is a white solid, was obtained.

¹H NMR (400 MHz, DMSO-D6) δ 8.41 (d, J=8 Hz, 2H), 6.68 (d, J=8 Hz, 2H), 4.22 (d, J=7.6 Hz, 4H), 4.01 (d, J=7.6 Hz, 2H), 2.41 (m, 2H), 1.27 (m, 1H), 0.53 (m, 2H), 0.44 (m, 2H).

LCMS: 189.2 [M].

### Example 71: 4-(Azetidin-1-yl)-1-(thiophen-3-ylmethyl)pyridinium bromide

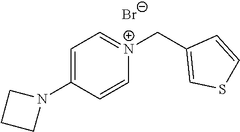 [SMILES: [Br-] ; c1cc(C[n+]2ccc(N3CCC3)cc2)cs1]

In the same manner as in Example 1, except that 4-(azetidin-1-yl)pyridine was used instead of 4-aminopyridine and 3-(bromomethyl)thiophene was used instead of (2-chloroethyl)benzene, 0.068 g (58%) of a desired compound, which is a white solid, was obtained.

¹H NMR (400 MHz, DMSO-D6) δ 8.37 (d. J=6.8 Hz, 2H), 7.63 (m, 2H), 7.61 (m, 2H), 7.16 (d, J=7.2 Hz, 2H), 6.68 (d, J=6.8 Hz, 2H), 5.35 (s, 2H), 4.21 (t, J=7.6 Hz, 4H), 2.41 (m, 2H).

LCMS: 231.1 [M].

### Example 72: 4-(Pyrrolidin-1-yl)-1-(selenophen-2-ylmethyl)pyridinium chloride

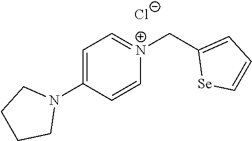 [SMILES: [Cl-] ; c1c[se]c(C[n+]2ccc(N3CCCC3)cc2)c1]

In the same manner as in Example 1, except that 4-(pyrrolidin-1-yl)pyridine was used instead of 4-aminopyridine and 2-(chloromethyl)selenophene was used instead of (2-chloroethyl)benzene, 0.058 g (15.8%) of a desired compound, which is a white solid, was obtained.

¹H NMR (400 MHz, DMSO-D6) δ 8.42 (d, J=7.2 Hz, 2H), 8.22 (d, J=5.6 Hz, 1H), 7.46 (d, J=3.6 Hz, 1H), 7.25 (d, J=5.6 Hz, 1H), 6.92 (d, J=7.2 Hz, 2H), 5.62 (s, 2H), 3.50 (m, 4H), 1.99 (m, 4H).

LCMS: 292.2 [M].

### Example 73: 4-Amino-1-(cyclopropylmethyl)pyrimidin-1-ium chloride

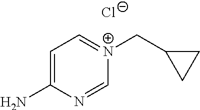 [SMILES: [Cl-] ; Nc1cc[n+](CC2CC2)cn1]

In the same manner as in Example 1, except that 4-aminopyrimidine was used instead of 4-aminopyridine and (2-chloromethyl)cyclopropane was used instead of (2-chloroethyl) benzene, 0.055 g (14%) of a desired compound, which is a white solid, was obtained.

¹H NMR (400 MHz, DMSO-D6) δ 9.20 (s, 1H), 9.04 (s, 1H), 8.86 (s, 1H), 8.38 (d, J=7.2 Hz, 1H), 6.87 (d, J=7.6 Hz, 1H), 3.98 (d, J=7.2 Hz, 1H), 1.30 (m, 1H), 0.53 (m, 2H), 0.47 (m, 2H).

LCMS: 150.2 [M].

### Example 74: 4-Amino-1-(selenophen-2-ylmethyl)pyrimidin-1-ium chloride

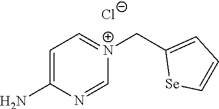 [SMILES: [Cl-] ; Nc1cc[n+](Cc2ccc[se]2)cn1]

In the same manner as in Example 1, except that 4-aminopyrimidine was used instead of 4-aminopyridine and 2-(chloromethyl)selenophene was used instead of (2-chloroethyl) benzene, 0.025 g (11.3%) of a desired compound, which is a white solid, was obtained.

¹H NMR (400 MHz, DMSO-D6) δ 9.36 (s, 1H), 9.16 (s, 1H), 9.03 (s, 1H), 8.36 (d, =7.2 Hz, 1H), 8.26 (d, J=7.2 Hz, 1H), 7.51 (d, J=2.4 Hz, 1H), 7.28 (d, J=5.6 Hz, 1H), 6.87 (d, J=7.2 Hz, 1H),

LCMS: 240.0 [M].

### Example 75: 4-Amino-1-(selenophen-2-ylmethyl)pyridazin-1-ium chloride

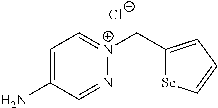 [SMILES: [Cl-] ; Nc1cc[n+](Cc2ccc[se]2)nc1]

In the same manner as in Example 1, except that 4-aminopyridazine was used instead of 4-aminopyridine and 2-(chloromethyl)selenophene was used instead of (2-chloroethyl) benzene, 0.019 g (8.66%) of a desired compound, which is a white solid, was obtained.

¹H NMR (400 MHz, DMSO-D6) δ 9.33 (s, 1H), 9.15 (s, 1H), 8.85 (s, 1H), 8.37 (d, 0.1-7.2 Hz, 1H), 8.23 (d, J=7.2 Hz, 1H), 7.50 (d, J=2.4 Hz, 1H), 7.23 (d, J=5.6 Hz, 1H), 6.89 (d, J=7.2 Hz, 1H).

LCMS: 240.0 [M].

### Example 76: 4-Amino-1-(selenophen-2-ylmethyl)pyridinium chloride

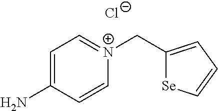 [SMILES: [Cl-] ; Nc1cc[n+](Cc2ccc[se]2)cc1]

In the same manner as in Example 1, except that 2-(chloromethyl)selenophene was used instead of (2-chloroethyl) benzene, 0.1 g (48%) of a desired compound, which is a white solid, was obtained.

¹H NMR (400 MHz, DMSO-D6) δ 8.33 (s, 2H), 8.31 (d, J=7.2 Hz; 2H), 8.23 (d, J=5.2 Hz, 1H), 7.44 (s, 1H), 7.27 (d, J=5.2 Hz, 1H), 6.88 (d, J=7.2 Hz, 2H), 5.59 (s, 2H).

LCMS: 239.0 [M].

### Example 77: 4-Amino-1-(thiophen-2-ylmethyl)pyridinium chloride

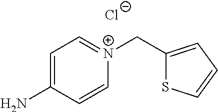 [SMILES: [Cl-] ; Nc1cc[n+](Cc2cccs2)cc1]

In the same manner as in Example 1, except that 2-(chloromethyl)thiophene was used instead of (2-chloroethyl) benzene, 0.37 g (37.4%) of a desired compound, which is a white solid, was obtained.

¹H NMR (400) MHz, DMSO-D6) δ 8.29 (m, 4H), 7.62 (d, J=4.8 Hz, 1H), 7.29 (s, 1H), 7.07 (m, 1H), 6.87 (d, J=7.2 Hz, 2H), 5.57 (s, 2H).

LCMS: 191.2 [M].

### Example 78: 1-(Furan-2-ylmethyl)-4-(pyrrolidin-1-yl)pyridinium chloride

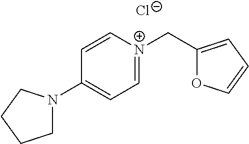 [SMILES: [Cl-] ; c1coc(C[n+]2ccc(N3CCCC3)cc2)c1]

In the same manner as in Example 1, except that 4-(pyrrolidin-1-yl)pyridine was used instead of 4-aminopyridine and 2-(chloromethyl)furan was used instead of (2-chloroethyl) benzene, 0.028 g (2%) of a desired compound, which is a white solid, was obtained.

¹H NMR (400 MHz, DMSO-D6) δ 8.30 (d, J=7.2 Hz, 2H), 7.69 (m, 2H), 6.90 (d, J=7.2 Hz, 2H), 6.63 (d, J=3.2 Hz, 1H), 6.48 (m, 1H), 5.43 (s, 2H), 3.48 (m, 4H), 1.98 (m, 4H).

LCMS: 229.1 [M].

### Example 79: 1-((5-Methylthiophen-2-yl)methyl)-4-(pyrrolidin-1-yl)pyridinium chloride

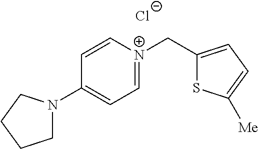 [SMILES: [Cl-] ; Cc1ccc(C[n+]2ccc(N3CCCC3)cc2)s1]

In the same manner as in Example 1, except that 4-(pyrrolidin-1-yl)pyridine was used instead of 4-aminopyridine and 2-(chloromethyl)-5-methylthiophene was used instead of (2-chloroethyl) benzene, 0.12 g (13%) of a desired compound, which is a white solid, was obtained.

¹H NMR (400 MHz. DMSO-D6) δ 8.36 (d, J=7.6 Hz, 2H), 7.10 (d, J=3.2 Hz, 1H), 6.90 (d, J=7.6 Hz, 2H), 6.74 (m, 1H), 5.51 (s, 2H), 3.49 (m, 4H), 1.99 (m, 4H).

LCMS: 259.1 [M].

### Example 80: 4-(Azetidin-1-yl)-1-(selenophen-3-ylmethyl)pyridinium chloride

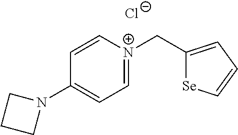 [SMILES: [Cl-] ; c1c[se]c(C[n+]2ccc(N3CCC3)cc2)c1]

In the same manner as in Example 1, except that 4-(azetidin-1-yl)pyridine was used instead of 4-aminopyridine and 2-(chloromethyl)selenophene was used instead of (2-chloroethyl) benzene, 0.12 g (40.7%) of a desired compound, which is a white solid, was obtained.

¹H NMR (400 MHz, DMSO-D6) δ 8.37 (d. J=7.2 Hz, 2H), 8.22 (m, 1H), 7.44 (m, 1H), 7.25 (m, 1H), 6.69 (d, J=7.2 Hz, 2H), 5.58 (s, 2H), 4.22 (t, J=8 Hz, 4H), 2.41 (m, 2H),

LCMS: 279.0 [M].

### Example 81: 2-Amino-4-(azetidin-1-yl)-1-(cyclopropylmethyl)pyridinium chloride

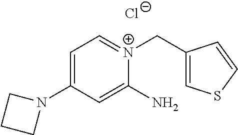 [SMILES: [Cl-] ; Nc1cc(N2CCC2)cc[n+]1Cc1ccsc1]

In the same manner as in Example 1, except that 4-(azetidin-1-yl)pyridin-2-amine was used instead of 4-aminopyridine and 2-(chloromethyl)thiophene was used instead of (2-chloroethyl) benzene, 0.16 g (79%) of a desired compound, which is a white solid, was obtained.

¹H NMR (400 MHz, DMSO-D6) δ 7.85 (d, J=7.6 Hz, 1H), 7.59 (m, 1H), 7.45 (m, 2H), 7.05 (m, 1H), 6.16 (m, 1H), 5.49 (m, 1H), 5.18 (s, 2H), 4.05 (m, 4H), 2.39 (m, 2H).

LCMS: 246.1 [M].

### Example 82: 2-Amino-4-(azetidin-1-yl)-1-(cyclopropylmethyl)pyridinium chloride

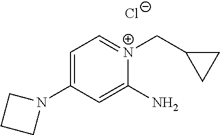 [SMILES: [Cl-] ; Nc1cc(N2CCC2)cc[n+]1CC1CC1]

In the same manner as in Example 1, except that 4-(azetidin-1-yl)pyridin-2-amine was used instead of 4-aminopyridine and (chloromethyl)cyclopropane was used instead of (2-chloroethyl) benzene, 0.11 g (45.6%) of a desired compound, which is a white solid, was obtained.

¹H NMR (300 MHz, DMSO-D6) δ 7.75 (d, J=7.5 Hz, 1H), 7.52 (s, 2H), 6.10 (m, 1H), 6.55 (m, 1H), 4.12 (m, 4H), 3.87 (d, J=7.2 Hz, 2H), 2.35 (m, 2H), 1.21 (m, 1H), 0.55 (m, 2H), 0.47 (m, 2H).

LCMS: 204.2 [M].

### Example 83: 2,4-Diamino-1-(cyclopropylmethyl)pyridinium chloride

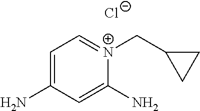 [SMILES: [Cl-] ; Nc1cc[n+](CC2CC2)c(N)c1]

In the same manner as in Examples 7 and 8, except that (chloromethyl)cyclopropane was used instead of (2-chloroethyl) benzene, 51 mg (51.8%) of a desired compound, which is a white solid, was obtained.

¹H NMR (300 MHz, DMSO-D6) δ 7.64 (d, J=7.2 Hz, 1H), 7.45 (s, 2H), 7.24 (s, 2H), 6.19 (m, 1H), 5.89 (s, 1H), 3.82 (d, J=7.2 Hz, 2H), 1.21 (m, 1H), 0.56 (m, 2H), 0.48 (m, 2H).

LCMS: 164.1 [M].

### Example 84: 2,4-Diamino-1-(4-chlorobenzyl)pyridinium chloride

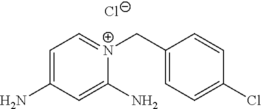 [SMILES: [Cl-] ; Nc1cc[n+](Cc2ccc(Cl)cc2)c(N)c1]

In the same manner as in Examples 7 and 8, except that 1-chloro-4-(chloromethyl)benzene was used instead of (2-chloroethyl) benzene, 46 mg (46.6%) of a desired compound, which is a white solid, was obtained.

¹H NMR (300 MHz, DMSO-D6) δ 7.67 (d, J=7.5 Hz, 1H), 7.41 (m, 4H), 7.17 (d, J=8.4 Hz, 2H), 6.20 (m, 1H), 5.85 (s, 1H), 5.18 (s, 2H).

LCMS: 234.2 [M].

### Example 85: 2-Amino-4-(azetidin-1-yl)-1-((5-methylthiophen-2-yl)methyl)pyridinium chloride

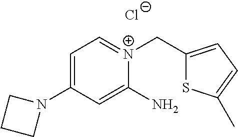 [SMILES: [Cl-] ; Cc1ccc(C[n+]2ccc(N3CCC3)cc2N)s1]

In the same manner as in Example 1, except that 4-(azetidin-1-yl)pyridin-2-amine was used instead of 4-aminopyridine and 2-(chloromethyl)-5-methylthiophene was used instead of (2-chloroethyl) benzene, 0.12 g (43%) of a desired compound, which is a white solid, was obtained.

¹H NMR (400 MHz, DMSO-D6) δ 7.85 (d, J=7.6 Hz, 1H), 7.59 (m, 1H), 7.45 (m, 2H), 7.05 (m, 1H), 6.16 (m, 1H), 5.49 (m, 1H), 5.18 (s, 2H), 4.05 (m, 4H), 2.41 (s, 3H), 2.39 (m, 2H).

LCMS: 260.1 [M].

### Example 86: 2-Amino-4-(azetidin-1-yl)-1-(selenophen-2-ylmethyl)pyridinium chloride

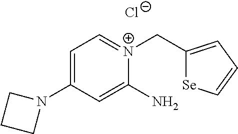 [SMILES: [Cl-] ; Nc1cc(N2CCC2)cc[n+]1Cc1ccc[se]1]

In the same manner as in Example 1, except that 4-(azetidin-1-yl)pyridin-2-amine was used instead of 4-aminopyridine and 2-(chloromethyl)selenophene was used instead of (2-chloroethyl) benzene, 0.09 g (38%) of a desired compound, which is a white solid, was obtained.

¹H NMR (300 MHz, DMSO-D6) δ 8.22 (d, J=5.6 Hz, 1H), 7.75 (d. J=7.5 Hz, 1H), 7.52 (s, 2H), 7.46 (d, J=3.6 Hz, 1H), 7.25 (d, J==5.6 Hz, 1H), 6.10 (m, 1H), 6.55 (m, 1H), 5.62 (s, 2H), 4.12 (m, 4H), 2.35 (m, 2H).

LCMS: 293.1 [M].

### Example 87: 2-Amino-4-(azetidin-1-yl)-1-benzylpyridinium chloride

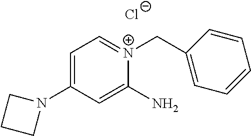 [SMILES: [Cl-] ; Nc1cc(N2CCC2)cc[n+]1Cc1ccccc1]

In the same manner as in Example 1, except that 4-(azetidin-1-yl)pyridin-2-amine was used instead of 4-aminopyridine and benzyl chloride was used instead of (2-chloroethyl) benzene, 0.1 g (48%) of a desired compound, which is a white solid, was obtained.

¹H NMR (300 MHz, DMSO-D6) δ 7.75 (d, J=7.5 Hz, 1H), 7.52 (s, 2H), 6.10 (m, 1H), 7.39 (m, 5H), 6.55 (m, 1H), 5.51 (s, 2H), 4.11 (m, 4H), 2.32 (m, 2H).

LCMS: 240.1 [M].

### Example 88: 2-Amino-1-benzyl-4-(pyrrolidin-1-yl)pyridinium chloride

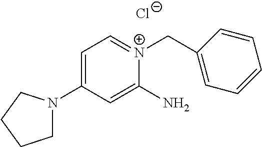 [SMILES: [Cl-] ; Nc1cc(N2CCCC2)cc[n+]1Cc1ccccc1]

In the same manner as in Example 1, except that 4-(pyrrolidin-1-yl)pyridin-2-amine was used instead of 4-aminopyridine and benzyl chloride was used instead of (2-chloroethyl) benzene, 0.15 g (30.8%) of a desired compound, which is a white solid, was obtained.

¹H NMR (300 MHz, DMSO-D6) δ 7.75 (d, J=7.5 Hz, 1H), 7.52 (s, 2H), 6.10 (m, 1H), 7.39 (m, 5H), 6.55 (m, 1H), 5.51 (s, 2H), 3.49 (m, 4H), 1.98 (m, 4H).

LCMS: 254.1 [M].

### Example 89: 2-Amino-1-(cyclopropylmethyl)-4-(pyrrolidin-1-yl)pyridinium chloride

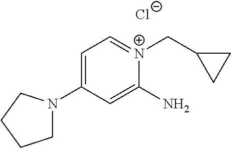 [SMILES: [Cl-] ; Nc1cc(N2CCCC2)cc[n+]1CC1CC1]

In the same manner as in Example 1, except that 4-(pyrrolidin-1-yl)pyridin-2-amine was used instead of 4-aminopyridine and (chloromethyl)cyclopropane was used instead of (2-chloroethyl) benzene, 0.18 g (14.6%) of a desired compound, which is a white solid, was obtained.

¹H NMR (300 MHz, DMSO-D6) δ 7.75 (d, J=7.5 Hz, 1H), 7.52 (s, 2H), 6.10 (m, 1H), 6.55 (m, 1H), 3.82 (d, J=7.2 Hz, 2H), 3.49 (m, 4H), 1.98 (m, 4H), 1.21 (m, 1H), 0.56 (m, 2H), 0.48 (m, 2H).

LCMS: 218.1 [M].

### Example 90: 2-Amino-1-((5-methylthiophen-2-yl)methyl)-4-(pyrrolidin-1-yl)pyridinium chloride

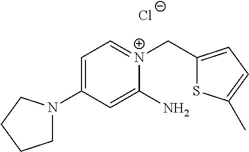 [SMILES: [Cl-] ; Cc1ccc(C[n+]2ccc(N3CCCC3)cc2N)s1]

In the same manner as in Example 1, except that 4-(pyrrolidin-1-yl)pyridin-2-amine was used instead of 4-aminopyridine and 2-(chloromethyl)-5-methylthiophene was used instead of (2-chloroethyl) benzene, 0.21 g (47.8%) of a desired compound, which is a white solid, was obtained.

¹H NMR (400 MHz, DMSO-D6) δ 7.85 (d, 0.1=7.6 Hz, 1H), 7.59 (m, 1H), 7.45 (m, 2H), 7.05 (m, 1H), 6.16 (m, 1H), 5.49 (m, 1H), 5.18 (s, 2H), 3.49 (m, 4H), 2.41 (s, 3H), 1.98 (m, 4H).

LCMS: 274.1 [M].

### Example 91: 2-Amino-4-(pyrrolidin-1-yl)-1-(selenophen-2-ylmethyl)pyridinium chloride

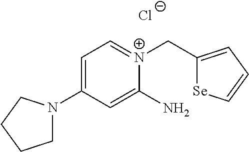 [SMILES: [Cl-] ; Nc1cc(N2CCCC2)cc[n+]1Cc1ccc[se]1]

In the same manner as in Example 1, except that 4-(pyrrolidin-1-yl)pyridin-2-amine was used instead of 4-aminopyridine and 2-(chloromethyl)selenophene was used instead of (2-chloroethyl) benzene, 0.17 g (38%) of a desired compound, which is a white solid, was obtained.

¹H NMR (300 MHz, DMSO-D6) δ 8.22 (d, J=5.6 Hz, 1H), 7.75 (d, J=7.5 Hz, 1H), 7.52 (s, 2H), 7.46 (d, J=3.6 Hz, 1H), 7.25 (d, J=5.6 Hz, 1H), 6.10 (m, 1H), 6.55 (m, 1H), 5.62 (s, 2H), 3.49 (m, 4H), 1.98 (m, 4H).

LCMS: 307.0 [M].

### Example 92: 2-Amino-1-(4-chlorobenzyl)-4-(pyrrolidin-1-yl)pyridinium chloride

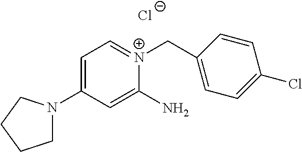 [SMILES: [Cl-] ; Nc1cc(N2CCCC2)cc[n+]1Cc1ccc(Cl)cc1]

In the same manner as in Example 1, except that 4-(pyrrolidin-1-yl)pyridin-2-amine was used instead of 4-aminopyridine and 4-chlorobenzyl chloride was used instead of (2-chloroethyl) benzene, 0.25 g (67.4%) of a desired compound, which is a white solid, was obtained.

¹H NMR (300 MHz, DMSO-D6) δ 7.75 (d. J=7.5 Hz, 1H), 7.52 (s, 2H), 7.41 (d. J=8.4 Hz, 2H), 7.17 (d, J=8.4 Hz, 2H), 6.10 (m, 1H), 6.55 (m, 1H), 5.51 (s, 2H), 3.49 (m, 4H), 1.98 (m, 4H).

LCMS: 288.1 [M].

### Example 93: 4-Amino-1-benzyl-2-ethoxypyridinium chloride

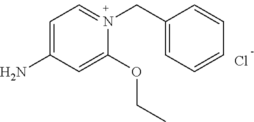

In the same manner as in Example 1, except that 2-ethoxylpyridin-4-amine was used instead of 4-amino pyridine and benzyl chloride was used instead of (2-chloroethyl) benzene, 0.12 g (31%) of a desired compound, which is a white solid, was obtained.

¹H NMR (300 MHz; DMSO-D6) δ 8.21 (s, 2H), 8.15 (d, J=7.2 Hz, 1H), 7.38 (m, 5H), 6.62 (m, 1H), 6.36 (m, 1H), 5.25 (s, 2H), 4.06 (q, J=7.2 Hz, 2H), 1.20 (t, J=7.2 Hz, 3H).

LCMS: 229.1 [M].

### Example 94: 4-Amino-1-benzyl-2-isopropoxypyridinium chloride

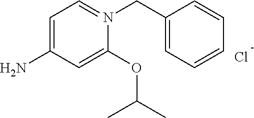

In the same manner as in Example 1, except that 2-isopropoxylpyridin-4-amine was used instead of 4-aminopyridine and benzyl chloride was used instead of (2-chloroethyl) benzene, 0.14 g (28%) of a desired compound, which is a white solid, was obtained.

¹H NMR (300 MHz, DMSO-D6) δ 8.20 (s, 2H), 8.13 (d, J=7.2 Hz, 1H), 7.38 (m, 5H), 6.62 (m, 1H), 6.36 (m, 1H), 5.25 (s, 2H), 4.04 (m, 1H), 1.38 (d, J=7.2 Hz, 6H).

LCMS: 243.1 [M].

### Example 95: 4-Amino-1-benzyl-2-cyclopropylpyridinium chloride

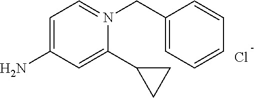

In the same manner as in Example 1, except that 2-cyclopropylpyridin-4-amine was used instead of 4-aminopyridine and benzyl chloride was used instead of (2-chloroethyl) benzene, 0.11 g (45.6%) of a desired compound, which is a white solid, was obtained.

¹H NMR (300 MHz, DMSO-D6) δ 8.21 (s, 2H), 8.11 (d, J=7.2 Hz, 1H), 7.39 (m, 5H), 6.60 (m, 1H), 6.33 (m, 1H), 5.26 (s, 2H), 1.50 (m, 1H), 0.56 (m, 2H), 0.48 (m, 2H).

LCMS: 225.1 [M].

### Example 96: 4-(Azetidin-1-yl)-1-benzyl-2-ethoxypyridinium chloride

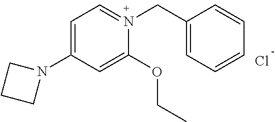

In the same manner as in Example 1, except that 4-(azetidin-1-yl)-2-ethoxypyridine was used instead of 4-aminopyridine and benzyl chloride was used instead of (2-chloroethyl) benzene, 0.15 g (30.8%) of a desired compound, which is a white solid, was obtained.

¹H NMR (300 MHz, DMSO-D6) δ 8.17 (d, J=7.2 Hz, 1H), 7.37 (m, 5H), 6.61 (m, 1H), 6.36 (m, 1H), 5.25 (s, 2H), 4.06 (q, J=7.2 Hz, 2H), 4.12 (m, 4H), 2.35 (m, 2H), 1.20 (t, J=7.2 Hz, 3H).

LCMS: 269.1 [M].

### Example 97: 4-(Azetidin-1-yl)-1-benzyl-2-isopropoxypyridinium chloride

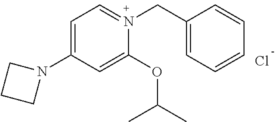

In the same manner as in Example 1, except that 4-(azetidin-1-yl)-2-isopropoxypyridine was used instead of 4-aminopyridine and benzyl chloride was used instead of (2-chloroethyl) benzene, 0.07 g (21%) of a desired compound, which is a white solid, was obtained.

¹H NMR (300 MHz, DMSO-D6) δ 8.15 (d. J=7.2 Hz, 1H), 7.38 (m, 5H), 6.62 (m, 1H), 6.36 (m, 1H), 5.25 (s, 2H), 4.12 (m, 4H), 4.04 (m, 1H), 2.35 (m, 2H), 1.38 (d, J=7.2 Hz, 6H).

LCMS: 283.1 [M].

### Example 98: 4-(Azetidin-1-yl)-1-benzyl-2-cyclopropylpyridinium chloride

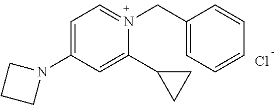

In the same manner as in Example 1, except that 4-(azetidin-1-yl)-2-cyclopropylpyridine was used instead of 4-aminopyridine and benzyl chloride was used instead of (2-chloroethyl) benzene, 0.05 g (42%) of a desired compound, which is a white solid, was obtained.

¹H NMR (300 MHz, DMSO-D6) δ 8.13 (d. J=7.2 Hz, 1H), 7.31 (m, 5H), 6.60 (m, 1H), 6.36 (m, 1H), 5.25 (s, 2H), 4.12 (m, 4H), 2.35 (m, 2H), 1.50 (m, 1H), 0.56 (m, 2H), 0.48 (m, 2H).

LCMS: 265.1 [M].

### Example 99: 1-Benzyl-2-ethoxy-4-(pyrrolidin-1-yl)pyridinium chloride

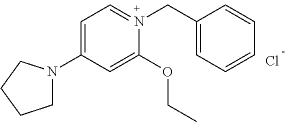

In the same manner as in Example 1, except that 4-(pyrrolidin-1-yl)-2-ethoxypyridine was used instead of 4-aminopyridine and benzyl chloride was used instead of (2-chloroethyl) benzene, 0.18 g (14.6%) of a desired compound, which is a white solid, was obtained.

¹H NMR (300 MHz, DMSO-D6) δ 8.17 (d, J=7.2 Hz, 1H), 7.37 (m, 5H), 6.61 (m, 1H), 6.36 (m, 1H), 5.25 (s, 2H), 4.06 (q, J=7.2 Hz, 2H), 3.49 (m, 4H), 1.98 (m, 4H), 1.20 (t, =7.2 Hz, 3H).

LCMS: 283.1 [M].

### Example 100: 1-Benzyl-2-isopropoxy-4-(pyrrolidin-1-yl)pyridinium chloride

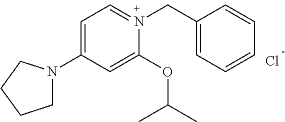

In the same manner as in Example 1, except that 4-(pyrrolidin-1-yl)-2-isopropoxypyridine was used instead of 4-aminopyridine and benzyl chloride was used instead of (2-chloroethyl) benzene, 0.13 g (38.1%) of a desired compound, which is a white solid, was obtained.

¹H NMR (300 MHz, DMSO-D6) δ 8.15 (d, J=7.2 Hz, 1H), 7.38 (m, 5H), 6.62 (m, 1H), 6.36 (m, 1H), 5.25 (s, 2H), 4.04 (m, 1H), 3.49 (m, 4H), 1.98 (m, 4H), 1.38 (d. J=7.2 Hz, 6H).

LCMS: 297.1 [M].

### Example 101: 1-Benzyl-2-cyclopropyl-4-(pyrrolidin-1-yl)pyridinium chloride

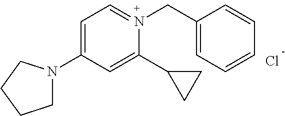

In the same manner as in Example 1, except that 4-(pyrrolidin-1-yl)-2-cyclopropylpyridine was used instead of 4-aminopyridine and benzyl chloride was used instead of (2-chloroethyl) benzene, 0.15 g (30.8%) of a desired compound, which is a white solid, was obtained.

¹H NMR (300 MHz, DMSO-D6) δ 8.13 (d, 0.1=7.2 Hz, 1H), 7.31 (m, 5H), 6.60 (m, 1H), 6.36 (m, 1H), 5.25 (s, 2H), 3.49 (m, 4H), 1.98 (m, 4H), 1.50 (m, 1H), 0.56 (m, 2H), 0.48 (m, 2H).

LCMS: 279.1 [M].

### Test Example 1: Measurement of Inhibitory Effect by Oxygen Consummation Rate and Extracellular Oxidation

The compounds synthesized by the methods disclosed in the Examples of the present invention have been measured on oxygen consumption rate and extracellular oxidation by the methods disclosed in the Test Examples below.

As the synthesized drugs inhibit oxidative phosphorylation and exhibit anti-cancer effects, Oxygen Consumption Rate (OCR) of cells for the compounds was measured.

3×10³ cells from A549 cell lines (purchased from ATCC-American Type Culture Collection), which are lung cancer cell lines, were placed on XF96 cell culture plates using RPMI1640 medium, and cultured at 37° C. in a 5% CO₂ condition for 16 hours or more for attachment.

After 16 hours, the cells were treated with the drug at six different concentrations between 0 μM and 20 μM. After 24 hours, the existing medium was removed, and XF assay medium (15 mM D-Glucose, 15 mM sodium pyruvate, 4 mM L-Glutamine, pH 7.4) was added. The cells were retreated with the drug, and additionally cultured in Prep station at 37° C. in a non-CO₂ condition for 1 hour. During the one-hour culture in the Prep station, a sensor cartridge was placed and calibrated for 20 minutes, and a plate with cells was placed to analyze the OCR. After the analysis was completed, XF96 plate was measured for cell viability using Cyquant assay, which measures the amount of intracellular DNA, in the following method. XF assay medium and the drug were removed, and the cells were placed in a cryogenic refrigerator (−80° C.) for at least 4 hours to be frozen. After the plate was made to be at room temperature, a solution where a lysis buffer and fluorescent GR dye were mixed was placed by 200 μL per well. After 20-minute reaction at room temperature, absorbance was measured between 480 nM to 520 nM to calculate cell viability. A measured value of a well untreated with the drug was converted to 100% by reflecting cell viability to the OCR value. Concentration of a drug which inhibits the OCR value reflecting cell viability by 50% was calculated.

TABLE 2

| Example | OCR IC₅₀ (μM) |
|---|---|
| Berberine | 4.6 |
| Example 1 | 1.1 |
| Example 7 | 0.4 |
| Example 8 | 1.2 |
| Example 11 | 0.7 |
| Example 12 | 0.8 |
| Example 13 | 16.5 |
| Example 15 | 2.3 |
| Example 16 | 1.5 |
| Example 17 | 4.2 |
| Example 18 | 0.6 |
| Example 19 | 0.5 |
| Example 20 | 1.8 |
| Example 21 | 0.9 |
| Example 22 | 7.9 |
| Example 23 | 0.9 |
| Example 24 | 0.8 |
| Example 25 | 1.6 |
| Example 26 | 2.4 |
| Example 27 | 0.4 |
| Example 28 | 0.4 |
| Example 29 | 5.3 |
| Example 30 | 0.9 |
| Example 31 | 4.3 |
| Example 32 | 0.3 |
| Example 33 | 1.5 |
| Example 34 | 1.1 |
| Example 35 | 0.5 |
| Example 36 | 0.7 |
| Example 37 | 1.1 |
| Example 38 | 1.1 |
| Example 39 | 2.7 |
| Example 40 | 0.9 |
| Example 41 | 2.7 |
| Example 42 | 1.5 |
| Example 43 | 0.9 |
| Example 44 | 1.1 |
| Example 45 | 5 |
| Example 46 | 0.8 |
| Example 47 | 5 |
| Example 48 | 0.8 |
| Example 49 | 2.4 |
| Example 50 | 1 |
| Example 51 | 3.1 |
| Example 52 | 0.8 |
| Example 53 | 0.5 |
| Example 54 | 1.1 |
| Example 55 | 3.1 |
| Example 56 | 0.8 |
| Example 57 | 0.5 |
| Example 58 | 2.5 |
| Example 59 | 2.8 |
| Example 60 | 3.3 |
| Example 61 | 15.7 |
| Example 62 | 1 |
| Example 63 | 7.1 |
| Example 64 | 10.2 |
| Example 66 | 5.2 |
| Example 68 | 0.8 |
| Example 70 | 3.4 |
| Example 71 | 3.4 |
| Example 72 | 1 |
| Example 75 | 18.8 |
| Example 76 | 1.5 |
| Example 77 | 2.4 |
| Example 78 | 2.1 |
| Example 79 | 0.8 |
| Example 80 | 1.3 |
| Example 81 | 2.1 |

### Test Example 2: Measurement of Inhibitory Effect of Cancer Cell Proliferation

The compounds prepared in the above Examples were evaluated for the inhibitory effect of cancer cell proliferation according to the method described in the following Test Example.

SK-MEL-28 cells derived from human melanoma were used, and the concentration (cell growth inhibitory concentration, IC₅₀) at which cell growth was inhibited to 50% was measured using MTT reagent (3-(4,5-dimethylthiazole-2-yl)-2,5-ditetrazolium bromide) to confirm the inhibitory effect of cancer cell proliferation of the drugs synthesized in Examples 1 to 84.

First, SK-MEL-28 cells were cultured in 96-well plates at a cell number of about 1,250 in RPMI-1640 medium containing 11.1 mM glucose and 10% calf blood serum or 0.75 mM glucose and 10% calf blood serum, and were cultured for 16 hours. Further, in order to determine the IC₅₀ value of each compound, the compound was added at a concentration of 1 mM, 200 μM, 40 μM, 8 μM, 1.6 μM, 0.32 μM, and 0.064 μM under the condition of 11.1 mM glucose, and 200 μM, 40 μM, 8 μM, 1.6 μM, 0.32 μM, 0.064 μM, and 0.0128 μM under the condition of 0.75 mM glucose in the well plate, and the well plate was cultured for 72 hours. After treatment of the compound, MTT was added to the culture medium to confirm living cells and further cultured for 2 hours. The resulting formazane crystal was dissolved using dimethyl sulfoxide, and the absorbance of the solution was measured at 555 nm. After culturing for 72 hours, the number of viable cells in the well plate treated with the compounds synthesized in the Examples relative to the number of cells cultured in the well plate without treatment of the compounds was expressed as cell viability (%) according to each treatment concentration. By using this, a cell viability curve graph was prepared, and the inhibitory effect of cancer cell proliferation was confirmed by calculating the concentration of the compound whose growth was inhibited to 50% (IC₅₀).

The results of the inhibitory effect of cancer cell growth are shown in Table 3 below.

TABLE 3

| | SK-MEL-28 11.1 mM | SK-MEL-28 0.75 mM |
|---|---|---|
| | glucose Cell viability | glucose Cell viability |
| Example | (IC₅₀ μM) | (IC₅₀ μM) |
| Example 1 | 163.4 | 17.9 |
| Example 7 | 16.8 | 20.7 |
| Example 8 | 75.1 | 4.7 |
| Example 11 | 38.5 | 11.9 |
| Example 12 | 55.5 | 12.8 |
| Example 13 | 319.7 | 129.3 |
| Example 14 | 277.4 | 154.4 |
| Example 15 | 47 | 6.8 |
| Example 16 | 28.2 | 8 |
| Example 17 | 75.4 | 6.9 |
| Example 18 | 16.9 | 10.7 |
| Example 19 | 8.8 | 5.9 |
| Example 20 | 143.4 | 14.7 |
| Example 21 | 27.4 | 12.5 |
| Example 22 | 192.8 | 18.5 |
| Example 23 | 51.1 | 5.4 |
| Example 24 | 16.8 | 4.4 |
| Example 25 | 40.6 | 15.5 |
| Exampie 26 | 193.8 | 102.3 |
| Example 27 | 19.5 | 1.7 |
| Example 28 | 3.8 | 0.7 |
| Example 29 | 162.5 | 38.5 |
| Example 30 | 23 | 8.2 |
| Example 31 | 418.5 | 56.2 |
| Example 32 | 38.8 | 8.1 |
| Example 33 | 101.6 | 7.7 |
| Example 34 | 101.2 | 7.9 |
| Example 35 | 7.5 | 5 |
| Example 36 | 13.1 | 2.8 |
| Example 37 | 10.8 | 1.5 |
| Example 38 | 11.7 | 1.5 |
| Example 39 | 76.5 | 45.3 |
| Example 40 | 30.6 | 7.5 |
| Example 41 | 234.3 | 35.1 |
| Example 42 | 198.7 | 35 |
| Example 43 | 45.5 | 14.4 |
| Example 44 | 104.7 | 15.2 |
| Example 45 | 81.4 | 16.6 |
| Example 46 | 49.6 | 7.9 |
| Example 47 | 166.6 | 20.3 |
| Example 48 | 46.3 | 19.9 |
| Example 49 | 54.5 | 10.3 |
| Example 50 | 207.5 | 13.9 |
| Example 51 | 201 | 29.1 |
| Example 52 | 366.7 | 40 |
| Example 53 | 76.4 | 8.7 |
| Example 54 | 29.7 | 3.4 |
| Example 55 | 45.9 | 0.1 |
| Example 56 | 118 | 4.4 |
| Example 57 | 23 | 1.1 |
| Example 58 | 93.9 | 3 |
| Example 59 | 33.4 | 2.8 |
| Example 60 | 24.1 | 20 |
| Example 61 | 42.,7 | 1.5 |
| Example 62 | 40.1 | 3.1 |
| Example 63 | 208.3 | 20.4 |
| Example 64 | 187.1 | 21 |
| Example 65 | 214.6 | 28.6 |
| Example 66 | 77 | 6.1 |
| Example 67 | 545.3 | 130.7 |
| Example 68 | 38.5 | 1.7 |
| Example 69 | 131.7 | 14.6 |
| Example 70 | 49.5 | 9.4 |
| Example 71 | 76.4 | 6.8 |
| Example 72 | 37.2 | 2.6 |
| Example 73 | 358.3 | 168.6 |
| Example 74 | 243 | 74.4 |
| Example 75 | 239.4 | 57.7 |
| Example 76 | 114 | 14.1 |
| Example 77 | 105.6 | 15.8 |
| Example 78 | 103.5 | 3.55 |
| Example 79 | 30.7 | 1.16 |
| Example 80 | 45.91 | 3.29 |
| Example 81 | 58.37 | 4.15 |

### Test Example 3: Test for Observing Antitumor Effect in Mouse Kidney Cancer Cells

RENCA, which are mouse kidney cancer cells, were cultured in RPMI 1640 medium containing 10% FBS and 1% anti-anti at 37° C. and 5% CO₂. 8- to 10-week-old BALB/c mice with a body weight range of 18 g to 20 g were subjected to a 7-day acclimation period, and then 1×10⁶/0.1 mL of RENCA cells in PBS were subcutaneously implanted on the right side of the backs of the mice. Seven days after implantation, group separation was performed based on the average of tumor volumes when the tumor volumes reached 50 mm² to 80 mm². A vehicle control group was intraperitoneally injected with PBS containing 2% DMSO and 2% Tween80, and an Example 62 administration group was intraperitoneally injected at a dose of 10 mg/kg, once a day for 2 weeks. Tumor volume measurements were performed twice weekly using Vemier calipers, and the volume of tumor was calculated by substituting long axis and short axis for 0.5×long axis×short axis². The results are shown in Table 4 and FIG. 1.

TABLE 4

| Days Post Tumor | | |
|---|---|---|
| Implantation | Vehicle | Example 62 |
| 7 | 81 ± 8 | 81 ± 7 |
| 9 | 160 ± 24 | 146 ± 17 |
| 12 | 380 ± 79 | 227 ± 39 |
| 14 | 615 ± 169 | 364 ± 68 |
| 16 | 970 ± 221 | 545 ± 132 |
| 20 | 1,687 ± 358 | 775 ± 206 |

From the results of the volume measurement of tumors, it was observed that the group to which Example 62 was administered remarkably inhibited tumor growth from the 6^{th} day of administration compared with the vehicle control group. The volume of tumor on day 14 which was the end day of observation yielded statistically significant data. Thus, Example 62 confirmed that there was a clear inhibitory effect on tumor growth in mouse kidney cancer cells.

1. A compound represented by Formula 1 below or a pharmaceutically acceptable salt thereof: 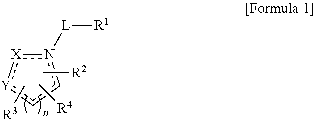
wherein, in Formula 1, [symbol] refers to a single bond or double bond, and a ring of Formula 1 comprises two to three double bonds, wherein the double bonds are not adjacent to each other,
X is CH, CNH₂, or N,
Y is CH, N, or S,
n is 1 or 2,
L is C₁₋₆ alkylene or C₁₋₆ alkenylene,
R¹ is C₆₋₁₄ aryl, C₅₋₂₀ heteroaryl, C₃₋₈ cycloalkyl, or C₃₋₈ heterocycloalkyl, and
R² to R⁴ are each independently hydrogen, amino (—NH₂), substituted amino (—NHR′ or —NR′R″), nitro, halogen, cyano, oxo, hydroxy, C₁₋₆ alkyl, C₃₋₈ cycloalkyl, C₃₋₈ heterocycloalkyl, C₁₋₆ alkoxy, C₁₋₆ haloalkyl, or C₁₋₆ haloalkoxy; or R² and R³ are positioned on adjacent carbon atoms and connected to each other to form a ring,
wherein R′ and R″ are each independently C₁₋₆ alkyl; or R′ and R″ are connected to each other to form a ring comprising a nitrogen atom to which R′ and R″ are bonded.

2. The compound of claim 1, wherein L is C₁₋₆ alkylene or C₁₋₆ alkenylene, which is unsubstituted or substituted with oxo.

3. The compound of claim 1, wherein R¹ is C₆₋₁₄ aryl, C₅₋₂₀ heteroaryl, C₃₋₈ cycloalkyl, or C₃₋₈ heterocycloalkyl, which is unsubstituted or substituted with one or more substituents selected from the group consisting of hydroxy, halogen, amino, cyano, nitro, C₁₋₆ alkyl, C₁₋₆ alkoxy, C₁₋₆ haloalkyl, and C₁₋₆ haloalkoxy.

4. The compound of claim 1, wherein R² to R⁴ are each independently C₁₋₆ alkyl, C₃₋₈ cycloalkyl, C₃₋₈ heterocycloalkyl, C₁₋₆ alkoxy, C₁₋₆ haloalkyl, or C₁₋₆ haloalkoxy, which are unsubstituted or substituted with one or more substituents selected from the group consisting of halogen, hydroxy, cyano, nitro, C₁₋₆ alkyl, C₁₋₆ alkoxy, C₁₋₆ haloalkyl, and C₁₋₆ haloalkoxy.

5. The compound of claim 1, wherein the ring formed by R² and R³ being positioned on adjacent carbon atoms and connected to each other is substituted with one or more substituents selected from the group consisting of halogen, hydroxy, cyano, nitro, C₁₋₆ alkyl, C₁₋₆ alkoxy, C₁₋₆ haloalkyl, and C₁₋₆ haloalkoxy.

6. The compound of claim 1, wherein, R′, R″, or the ring formed when R′ and R″ are connected to each other, which comprises a nitrogen atom to which R′ and R″ are bonded, is each independently substituted with one or more substituents selected from the group consisting of hydroxy, cyano, nitro, C₁₋₆ alkyl, C₁₋₆ alkoxy, C₁₋₆ haloalkyl, and C₁₋₆ haloalkoxy.

7. The compound of claim 1, wherein the ring formed by R″ and R³ being positioned on adjacent carbon atoms and connected to each other; or the ring comprising a nitrogen atom to which R′ and R″ are bonded, formed by R′ and R″ being connected to each other, is C₆₋₁₄ aryl, C₅₋₂₀ heteroaryl, C₃₋₁₀ cycloalkyl, or C₃₋₁₀ heterocycloalkyl.

8. The compound of claim 1, wherein L is methylene, ethylene, propylene, or —CH₂—C(O)—.

9. The compound of claim 1, wherein R¹ is C₆₋₈ aryl, C₃₋₈ cycloalkyl, or C₅₋₈ heteroaryl.

10. The compound of claim 1, wherein R¹ is phenyl, cyclopropyl, cyclobutyl, cyclopentyl, cyclohexyl, thiophene, furan, or selenophene.

11. The compound of claim 1, wherein R¹ is C₆₋₈ aryl, C₃₋₈ cycloalkyl, or C₅₋₈ heteroaryl, which is unsubstituted or substituted with halogen, C₁₋₆ haloalkoxy, or C₁₋₆ alkyl.

12. The compound of claim 1, wherein R¹ is C₆₋₈ aryl, C₃₋₈ cycloalkyl, or C₅₋₈ heteroaryl, which is unsubstituted or substituted with chlorine, fluorine, trifluoromethoxy, or methyl.

13. The compound of claim 1, wherein R² to R⁴ are each independently hydrogen, amino (—NH₂), substituted amino (—NHR′ or —NR′R″), oxo, nitro, halogen, C₃₋₈ cycloalkyl, C₁₋₆ alkyl, or C₁₋₆ alkoxy.

14. The compound of claim 1, wherein R² to R⁴ are each independently hydrogen, amino (—NH₂), substituted amino (—NHR′ or —NR′R″), oxo, nitro, halogen, cyclopropyl, methyl, methoxy, ethoxy, or isopropoxy.

15. The compound of claim 1, wherein R′ and R″ are each independently C₁₋₆ alkyl; or R′ and R″ are connected to each other to form C₃₋₁₀ heterocycloalkyl comprising a nitrogen atom to which R′ and R″ are bonded.

16. The compound of claim 1, wherein R′ and R″ are each independently methyl, tertiary butyl,

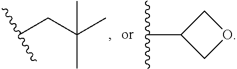

17. The compound of claim 15, wherein C₃₋₁₀ heterocycloalkyl comprising a nitrogen atom to which R′ and R″ are bonded, formed by R′ and R″ being connected to each other, is morpholinyl, azetidinyl, pyrrolidinyl, piperidinyl, or azepanyl, which is unsubstituted or substituted with one or more halogens.

18. The compound of claim 1, wherein R² and R³ are positioned on adjacent carbon atoms and connected to each other to form C₆₋₈ aryl, C₃₋₁₀ cycloalkyl, or C₃₋₁₀ heterocycloalkyl.

19. The compound of claim 18, wherein C₃₋₁₀ cycloalkyl formed by R² and R³ being positioned on adjacent carbon atoms and connected to each other is cyclohexyl.

20. The compound of claim 18, wherein C₃₋₁₀ heterocycloalkyl formed by R² and R³ being positioned on adjacent carbon atoms and connected to each other is piperidinyl or morpholinyl.

21. The compound of claim 18, wherein C₆₋₈ aryl formed by R² and R³ being positioned on adjacent carbon atoms and connected to each other is benzo.

22. The compound of claim 1, wherein the compound is selected from the group consisting of:
1) 4-amino-1-phenethylpyridinium chloride;
2) 4-nitro-1-phenethyl-1H-imidazole;
3) 4-nitro-1-phenethyl-1H-imidazole hydrochloride;
4) 3-nitro-1-phenethyl-1H-pyrazole;
5) 1-phenethyl-1H-pyrazol-3-amine;
6) 6-amino-3-phenethylpyrimidin-4(3H)-one;
7) 4-amino-2-bromo-1-phenethylpyridinium chloride;
8) 2,4-diamino-1-phenethylpyridinium bromide;
9) 1-phenethyl-1H-imidazole;
10) 2,6-diamino-3-phenethylpyrimidin-4(3H)-one;
11) 4-amino-1-(2-chlorophenethyl)pyridinium chloride;
12) 2,4-diamino-1-(2-chlorophenethyl)pyridinium bromide;
13) 3-phenethylthiazol-3-ium iodide;
14) 2-amino-3-phenethylthiazol-3-ium iodide;
15) 4-amino-2-cyclopropyl-1-phenethylpyridinium iodide;
16) 4-amino-1-phenethylquinolinium iodide;
17) 4-(dimethylamino)-1-phenethylpyridinium chloride;
18) 4-amino-2-fluoro-1-phenethylpyridinium chloride;
19) 4-amino-1-(3,4-dichlorophenethyl)pyridinium chloride;
20) 4-amino-1-benzylpyridinium chloride;
21) 4-amino-1-benzyl-2-fluoropyridinium chloride;
22) 1-phenethyl-5,6,7,8-tetrahydroquinolinium chloride;
23) 4-amino-1-(3-phenylpropyl)pyridinium chloride;
24) 4-amino-2-fluoro-1-(3-phenylpropyl)pyridinium chloride;
25) 4-amino-1-(2-oxo-2-(4-(trifluoromethoxy)phenyl)ethyl)pyridinium bromide;
26) 4-amino-1-(2-oxo-2-phenylethyl)pyridinium bromide
27) 4-amino-1-(2-cyclohexylethyl)pyridinium bromide;
28) 4-amino-1-(2-cyclohexylethyl)-2-fluoropyridinium bromide;
29) 2,4-diamino-1-benzylpyridinium chloride;
30) 4-amino-1-benzyl-2-chloropyridinium chloride;
31) 4-amino-1-(cyclopropylmethyl)pyridinium chloride;
32) 4-amino-2-chloro-1-phenethylpyridinium chloride;
33) 4-(methylamino)-1-phenethylpyridinium chloride;
34) 1-benzyl-4-(methylamino)pyridinium chloride;
35) 4-amino-1-(3,4-dichlorobenzyl)-2-fluoropyridinium chloride;
36) 4-amino-1-(3,4-dichlorobenzyl)pyridinium chloride;
37) 1-(3,4-dichlorobenzyl)-4-(methylamino)pyridinium chloride;
38) 1-(3,4-dichlorobenzyl)-4-(dimethylamino)pyridinium chloride;
39) 4-amino-1-(cyclopropylmethyl)-2-fluoropyridinium chloride;
40) 2,4-diamino-1-(2-cyclohexylethyl)pyridinium bromide;
41) 1-(cyclopropylmethyl)-4-(methylamino)pyridinium chloride;
42) 1-(cyclopropylmethyl)-4-(dimethylamino)pyridinium chloride;
43) 4-amino-3-methyl-1-benzylpyridinium chloride;
44) 4-amino-3-methyl-1-phenethylpyridinium chloride;
45) 4-amino-1-benzyl-2-methoxypyridinium chloride;
46) 4-amino-1-(cyclohexylmethyl)pyridinium bromide;
47) 4-amino-1-(cyclobutylmethyl)pyridinium bromide;
48) 4-amino-1-(cyclobutylmethyl)-2-fluoropyridinium bromide;
49) 4-amino-1-(4-fluorobenzyl)pyridinium bromide;
50) 1-benzyl-4-morpholinopyridinium chloride;
51) 4-morpholino-1-phenethylpyridinium chloride;
52) 4-morpholino-1-(cyclopropylmethyl)pyridinium chloride;
53) 1-(2-cyclohexylethyl)-4-morpholinopyridinium bromide;
54) 1-benzyl-4-(pyrrolidin-1-yl)pyridinium chloride;
55) 1-phenethyl-4-(pyrrolidin-1-yl)pyridinium chloride;
56) 1-(cyclopropylmethyl)-4-(pyrrolidin-1-yl)pyridinium chloride;
57) 1-(cyclohexylmethyl)-4-(pyrrolidin-1-yl)pyridinium bromide;
58) 1-(cyclobutylmethyl)-4-(pyrrolidin-1-yl)pyridinium chloride;
59) 1-benzyl-4-(piperidin-1-yl)pyridinium chloride;
60) 4-(azepan-1-yl)-1-benzylpyridinium chloride;
61) 1-benzyl-4-(neopentylamino)pyridinium chloride;
62) 4-(pyrrolidin-1-yl)-1-(thiophen-3-ylmethyl)pyridinium bromide;
63) 6-(cyclopropylmethyl)-1,2,3,4-tetrahydro-1,6-naphthyridin-6-ium chloride;
64) 6-(cyclopropylmethyl)-2,3-dihydro-1H-pyrido[3,4-b][1,4]oxazin-6-ium chloride;
65) 1-benzyl-4-(4,4-difluoropiperidin-1-yl)pyridinium chloride;
66) 4-(azetidin-1-yl)-1-benzylpyridinium chloride;
67) 1-benzyl-4-(oxetan-3-ylamino)pyridinium chloride;
68) 4-(pyrrolidin-1-yl)-1-(thiophen-2-ylmethyl)pyridinium chloride;
69) 1-benzyl-4-(tert-butylamino)pyridinium chloride;
70) 4-(azetidin-1-yl)-1-(cyclopropylmethyl)pyridinium chloride;
71) 4-(azetidin-1-yl)-1-(thiophen-3-ylmethyl)pyridinium bromide;
72) 4-(pyrrolidin-1-yl)-1-(selenophen-2-ylmethyl)pyridinium chloride;
73) 4-amino-1-(cyclopropylmethyl)pyrimidin-1-ium chloride;
74) 4-amino-1-(selenophen-2-ylmethyl)pyrimidin-1-ium chloride;
75) 4-amino-1-(selenophen-2-ylmethyl)pyridazin-1-ium chloride;
76) 4-amino-1-(selenophen-2-ylmethyl)pyridinium chloride;
77) 4-amino-1-(thiophen-2-ylmethyl)pyridinium chloride;
78) 1-(furan-2-ylmethyl)-4-(pyrrolidin-1-yl)pyridinium chloride;
79) 1-((5-methylthiophen-2-yl)methyl)-4-(pyrrolidin-1-yl)pyridinium chloride;
80) 4-(azetidin-1-yl)-1-(selenophen-3-ylmethyl)pyridinium chloride;
81) 2-amino-4-(azetidin-1-yl)-1-(cyclopropylmethyl)pyridinium chloride;
82) 2-amino-4-(azetidin-1-yl)-1-(cyclopropylmethyl)pyridinium chloride;
83) 2,4-diamino-1-(cyclopropylmethyl)pyridinium chloride;
84) 2,4-diamino-1-(4-chlorobenzyl)pyridinium chloride;
85) 2-amino-4-(azetidin-1-yl)-1-((5-methylthiophen-2-yl)methyl)pyridinium chloride;
86) 2-amino-4-(azetidin-1-yl)-1-(selenophen-2-ylmethyl)pyridinium chloride;
87) 2-amino-4-(azetidin-1-yl)-1-benzylpyridinium chloride;
88) 2-amino-1-benzyl-4-(pyrrolidin-1-yl)pyridinium chloride;
89) 2-amino-1-(cyclopropylmethyl)-4-(pyrrolidin-1-yl)pyridinium chloride;
90) 2-amino-1-((5-methylthiophen-2-yl)methyl)-4-(pyrrolidin-1-yl)pyridinium chloride;
91) 2-amino-4-(pyrrolidin-1-yl)-1-(selenophen-2-ylmethyl)pyridinium chloride;
92) 2-amino-1-(4-chlorobenzyl)-4-(pyrrolidin-1-yl)pyridinium chloride;
93) 4-amino-1-benzyl-2-ethoxypyridinium chloride;
94) 4-amino-1-benzyl-2-isopropoxypyridinium chloride;
95) 4-amino-1-benzyl-2-cyclopropylpyridinium chloride;
96) 4-(azetidin-1-yl)-1-benzyl-2-ethoxypyridinium chloride;
97) 4-(azetidin-1-yl)-1-benzyl-2-isopropoxypyridinium chloride;
98) 4-(azetidin-1-yl)-1-benzyl-2-cyclopropylpyridinium chloride;
99) 1-benzyl-2-ethoxy-4-(pyrrolidin-1-yl)pyridinium chloride;
100) 1-benzyl-2-isopropoxy-4-(pyrrolidin-1-yl)pyridinium chloride; and
101) 1-benzyl-2-cyclopropyl-4-(pyrrolidin-1-yl)pyridinium chloride.

23. A method for preparing the compound of claim 1 or a pharmaceutically acceptable salt thereof, comprising reacting a compound represented by Formula 2 below and a compound represented by Formula 3 below: 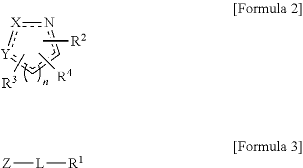
wherein, in Formulas 2 and 3, Z is halogen, and [symbol] , X, Y, n, L, R¹, R², R³, and R⁴ are the same as defined in claim 1.

24. A pharmaceutical composition, comprising the compound of claim 1 or a pharmaceutically acceptable salt thereof.

25. The composition of claim 24, wherein the composition is for anti-cancer use.

26. The composition of claim 24, wherein the composition further comprises an anti-cancer agent.

27. The composition of claim 24, wherein the cancer is one or more selected from the group consisting of uterine cancer, breast cancer, gastric cancer, brain cancer, rectal cancer, colorectal cancer, lung cancer, skin cancer, blood cancer, pancreatic cancer, renal cancer, bladder cancer, prostate cancer, and liver cancer.

28. A method for treating or preventing cancer, comprising administering a therapeutically effective amount of the pharmaceutical composition of claim 24 to a subject in need thereof.

29. The method of claim 28, wherein the method is to administer the pharmaceutical composition alone or in combination with an anti-cancer agent.

30. The method of claim 28, wherein the cancer is one or more selected from the group consisting of uterine cancer, breast cancer, gastric cancer, brain cancer, rectal cancer, colorectal cancer, lung cancer, skin cancer, blood cancer, pancreatic cancer, renal cancer, bladder cancer, prostate cancer, and liver cancer.
